SEQUENCE LISTING (4 sequences)

SEQ ID NO: 1 - DNA, 636 bp, homo sapiens
  CDS (1)..(636)
atgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctga

SEQ ID NO: 2 - protein, 211 aa, homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 3 - DNA, 5418 bp, Artificial
  recombinant adeno-associated viral vector, including plasmid back bone, AAV2 internal terminal repeats, CMV promoter, human TIMP-3 cDNA, and bovine growth hormone polyadenylation signal
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 4 - protein, 211 aa, Artificial; identical to SEQ ID NO: 2
  recombinant adeno-associated viral vector, including plasmid back bone, AAV2 internal terminal repeats, CMV promoter, human TIMP-3 cDNA, and bovine growth hormone polyadenylation signal

(19) United States
(12) Patent Application Publication
(10) Pub. No.: US 2004/0248826 A1
(43) Pub. Date: Dec. 9, 2004

(54) Treatment of cancer by in vivo gene-transfer induced TIMP-3 expression

(76) Inventors: Alberto Auricchio, Naples (IT); Markus Hildinger, Boston, MA (US)

Correspondence Address: MARKUS HILDINGER, ONE DEVONSHIRE PLACE, APT. 3401, BOSTON, MA, 02109, US

(21) Appl. No.: 10/452,878

(22) Filed: Jun. 3, 2003

(51) Int. Cl. A61K 48/00

(52) U.S. Cl. 514/44; 424/93.2

(57) ABSTRACT

Cancer is a major cause of death in developed countries. Yet, there is still a high degree of unmet need in the prevention, treatment and/or cure of cancer. The present invention relates to methods for treating cancer, preventing cancer and/or inhibiting the growth of cancer (cells) by administering to a mammalian subject a gene transfer vector in vivo comprising a nucleic acid composition whose expression directly or indirectly leads to the expression and/or secretion of Tissue inhibitor of metalloproteinase-3 (TIMP-3). Upon successful transduction, expression and/or secretion of TIMP-3—either locally (in the vicinity or within the cancer cells) or systemically—will inhibit cancer growth. Also provided are pharmaceutical kits containing the gene transfer vector in a suitable pharmaceutical suspension for administration.

### BACKGROUND OF THE INVENTION

1. Field of the Invention

The present invention relates to methods of treating neoplastic disease such as (solid) tumors in general and lung cancer in particular by means of gene transfer induced expression of Tissue inhibitor of metalloproteinase-3 (TIMP-3) or a substantially homologous protein. More specifically, the invention relates to the use of gene transfer vectors in general and to recombinant adeno-associated viral vectors in particular, to deliver to and/or within a mammalian subject nucleic acid compositions in vivo that encode one or a combination of the following:

(a) TIMP-3 or a substantially homologous protein

(b) proteins (such as e.g., transcription factors) that result in the expression of TIMP-3

(c) nucleic acids (such as e.g., RNA) that result in the expression of TIMP-3.

2. Background of the Invention

A neoplasm, or tumor, is a neoplastic mass resulting from abnormal uncontrolled cell growth, which may cause swelling on the body surface, and which can be benign or malignant. Benign tumors generally remain localized. Malignant tumors are collectively termed cancers. The term “malignant” generally means that the tumor can invade and destroy neighboring body structures and spread to distant sites to cause death (for review, see [1]). Treatment options, such as surgery, chemotherapy and radiation treatment, are either ineffective or present serious side effects. Thus, there is a need to develop new therapeutics for the treatment of cancer.

The present invention offers a novel and useful method for treating cancer, preventing cancer and/or inhibiting the growth of cancer (cells) by administering to a mammalian subject a gene transfer vector in vivo comprising a nucleic acid composition whose expression directly or indirectly leads to the expression of Tissue Inhibitor of metalloproteinase-3 (TIMP-3) or a substantially homologous protein. Upon successful transduction, expression and secretion of TIMP-3—either locally (in the vicinity or within the cancer cells) or systemically—will inhibit cancer growth. Also provided are pharmaceutical kits containing the gene transfer vector in a suitable pharmaceutical suspension for administration.

The inventors are the first to demonstrate the anti-cancer effect of expressing TIMP-3 by administering a gene transfer vector in vivo. Gene transfer induced expression of TIMP-3 offers advantages over existing treatments: The deficient activity of many antitumoral active compounds can be explained, at least to some degree, by the fact that the tumor cells within a tumor node are inaccessible to the antitumoral, in particular high molecular weight, active compounds [2], [3, 4]. Moreover, many tumor cells develop resistance to current treatment modalities. Gene transfer induced expression of TIMP-3 overcomes these limitations as its prime target of action are not the tumor cells themselves, but the surrounding (non-cancerous) tissue and the inhibition of ECM degradation and neovascularization of tumors (angiogenesis). As ECM degradation and angiogenesis play a crucial role in the survival of tumors, interference with these processes through expression and secretion of TIMP-3 results in the inhibition of cancer growth—as demonstrated by the inventors.

In addition, TIMP-3 combines three anti-cancer effects—an advantage over other treatment methods currently in development that focus mostly only on one of these targets:

(1) anti-angiogenic activity (and thus inhibition of neovascularization)

(2) inhibition of MMPs (and thus inhibition of ECM degradation and metastasis)

(3) anti-apoptotic activity.

Besides, compared to other anti-cancer therapies such as small molecules or protein therapies, the gene transfer-induced expression of TIMP-3 guarantees both a high local concentration of the active factor close to the tumor and continuous presence of the active factor due to constitutive synthesis.

Last, given the alternative treatment paradigm of TIMP-3 compared to e.g., cytotoxic drugs or radiation therapy, gene transfer induced expression of TIMP-3 might function in addition to current treatments to increase overall treatment efficacy.

### 3 DESCRIPTION OF PRIOR ART

3.1 Angiogenesis

Angiogenesis is the process by which new capillaries are formed by sprouting from pre-existing vessels. It is regulated through the combined action of pro-angiogenic and anti-aniogenic factors [5] [6] [7].

Pro-angiogenic factors favor angiogenesis. They can be classified into

(a) cytokines and growth factors such as

Fibroblast Growth Factor (FGF)

Vascular Endothelial Growth Factor (VEGF)

TGF.alpha and TGF.beta

angiopoietins

Interleukins (IL-1, IL-6, IL-8)

GM-CSF

PDGF

PDECGF

EGF

TNF.alpha

HGF

(b) cell surface receptors such as

Flk1 (=VEGFR2;=KDR; VEGF receptor)

Flt1 (=VEGFR1; VEGF receptor; [8])

Neurophilin

(c) Integrins such as

.alpha.v.beta.3 (whose expression is induced by, e.g., TNF.alpha, FGF-2, GM-CSF etc.)

.alpha.v.beta.5

(d) proteases such as matrix metalloproteases (MMPs)

(e) small molecules (e.g., retinoic acid; Cu)

Within the FGF family, especially FGF-1 and FGF-2 are said to act synergistically with VEGF in angiogenesis. The VEGF family comprises at least 5 members with VEGF-A having the strongest angiogenic activity. It is also expressed by different tumors (including lung tumors); more generally, its secretion is induced by hypoxia. Thus, VEGF is a potent enhancer of angiogenesis and vascular permeability in vivo and has been shown to be crucial to vasculogenesis and angiogenesis. Many tumor cells actively produce VEGF. The biological effects of VEGF are mediated by binding of one of the isoforms of VEGF to members of the VEGF receptor (VEGFR) family, Flk1 or Flt1, in combination with integrin alpha.v.beta.3 in case of Flk1. Flk1 and Flt1 are receptor tyrosine kinases; Flk1 is the primary receptor for VEGF-A whereas Flt1 can also bind VEGF-A homologs (e.g., VEGF-B, PIGF).

Remodeling of the extracellular matrix (ECM) is an important regulator of neovascularization, vascular morphogenesis and vascular invasion. The initiating stage in angiogenesis is believed to be a proteolytic degradation of the capillary basement membrane by (matrix) metalloproteinases (MMPs) and serine proteases. Members of the MMP family have an important role in angiogenesis; thus, mice deficient in MMP2 and MMP9 show reduced angiogenesis in vivo.

Anti-angiogenic factors inhibit angiogenesis. They can be classified into

(a) cytokines and growth factors such as

troponin

Platelet Factor 4

TGF.beta.1

Interferon.alpha and Interferon.beta

somatostatin

(b) proteolytic fragments such as

angiostatin

endostatin (collagen XVIII fragment)

thrombospondin fragment

fibronectin fragments

vasostatin (kallikrein fragment)

(c) Tissue inhibitors of metalloproteases (TIMPs)

(d) small molecules such as some retinoids, zinc or 2-methoxyestradiol

(e) other proteins such as laminin or CM101

3.2 Tissue Inhibitors of Metalloproteinases

It has been published that the activities of MMPs in normal tissues are thought to be regulated by the presence of endogenous tissue inhibitors of MMPs, TIMPs [9] [10] [11]. The ratio of the amounts of TIMP and MMPs is thought to maintain a balance between the rates of degradation and synthesis of ECM. The TIMP/MMP balance is involved in such pathologic processes as arthritis, atherosclerosis and tumor metastasis [12].

The family of endogenous MMP inhibitors currently comprises five members (TIMP1 through TIMP5). By virtue of their MMP-inhibitory activity, TIMP family members have a potentially important function in regulating matrix composition and thereby affect a wide range of physiological processes including cell growth, invasion, migration, angiogenesis, transformation and apoptosis.

All members of the TIMP-family comprise approximately 190 amino acids which are arranged into two domains. Each domain contains three disulfide bridges with the N-terminal domain harboring most of the biologic function [13].

TIMP3 [14, 15] [16] is an important member of the TIMP-family. It is the only TIMP with high affinity to the ECM and shows apoptotic activity (compared to the anti-apoptotic activity of TIMP-1 [17] and TIMP-3) [18] [19]. It is an ubiquitously expressed ECM-bound protein and a potent inhibitor of angiogenesis by inhibiting

(a) MMPs

(b) ADAMs (a disintegrin and a metalloproteinase domain), such as ADAM 17, ADAM 10 and ADAM 12S

(c) ADAMTSs (ADAM with thrombospondin-like repeats), such as ADAMTS 4 and ADAMTS 5

(d) Shedding of IL-6, L-selectin, Syndecan 1 and Syndecan 4.

Mutations in TIMP3 are associated with Sorsby fundus dystrophy [20], a macular degenerative disease manifested by sudden loss of visual acuity in the third to fourth decades of life due to choroidal (submacular) neovascularization.

3.2.1 TIMP-3 and Cancer Treatment

TIMP-3 combines three anti-cancer effects, which make it interesting for therapeutic purposes:

(1) anti-angiogenic activity (and thus inhibition of neovascularization)

(2) inhibition of MMPs (and thus inhibition of ECM degradation and metastasis)

(3) anti-apoptotic activity.

Anand-Apte et al. were the first to prove anti-tumor activity of TIMP-3 by using naked DNA [16] and in vitro transfection of tumor cells. More specifically, they transfected breast carcinoma and malignant melanoma cell with TIMP-3 expression plasmids and injected the cells subcutaneously into nude mice. Growth curves of the resulting tumors over a period of 6 to 8 weeks demonstrated that increased expression of TIMP-3 resulted in a statistically significant suppression of tumor growth. Later, two other studies confirmed the anti-tumoral activity of TIMP-3 in vitro in tissue culture systems [21, 22]. Ahonen et al. also provided data in an in vitro gene transfer study where the adenoviral delivery of the TIMP-3 gene to human melanoma (A2058) and SCC (UT-SCC-7) cells in vitro inhibited tumorigenesis after subcutaneous (s.c.) injection of the infected cells into SCID/SCID mice [23]. Similar results were obtained by Spurbeck et al. [24] using retroviral in vitro TIMP-3 gene transfer to effect sustained autocrine expression of TIMP-3 in murine neuroblastoma and melanoma tumor cells. The same group reproduced its earlier findings by coinjecting retroviral TIMP-3 vector-producer cells with tumor cells in a mouse model [25].

The present invention distinguishes itself from prior art in several aspects and proves itself as novel, non-obvious and as a useful advancement in the art:

(1) All of the prior art studies listed did not use in vivo delivery of the gene transfer vector, but either relied on ex vivo or in vitro transduction or transfection of cells (which then subsequently were or were not readministered to a mammalian subject). In that respect, the present invention is novel and a scientific advancement as it allows for in vivo administration of the gene transfer vector.

(2) Moreover, prior studies did not show a preventive effect, but only a palliative effect whereas the present invention shows both a preventive and palliative effect, as demonstrated in its preferred embodiment (AAV-mediated TIMP-3 expression in a lung cancer model).

(3) Additionally, most of prior art approaches require a direct transfection or transduction of tumor cells; the present invention overcomes this limitation as shown in its preferred embodiment (the prevention and treatment of lung metastases by in vivo administration of an AAV2/5 adeno-associated viral vector comprising a TIMP-3 expression cassette), where non-malignant lung cells were first transduced with recombinant AAV2/5 adeno-associated viral vector comprising a TIMP-3 expression cassette, and some days later, tumor cells were administered to the mammalian subject.

(4) Besides, compared to direct injection of retroviral producer cells [25], the present invention represents another advantage as it does not require the injection of cellular material, but allows for direct in vivo administration of the gene transfer vector—an important safety aspect as it allows for more exact dosing and makes the approach clinically superior: It might be questionable if the injection of non-self viral producer cells into human subjects will be clinically approved; and even if approved, one can expect an immune response against the non-self retroviral producer cells which subsequently will be destroyed, thus limiting any potential therapeutic effect.

(5) Last, prior art has not yet demonstrated the anti-tumoral activity of TIMP-3 in lung cancer treatment in general, and a preventive treatment paradigm for lung cancer in particular. Thus, the present invention is the first study to prove the preventive and palliative anti-lung cancer activity of TIMP-3.

Thus, the present invention is novel, non-obvious and a useful advancement of the art.

3.3 Metalloproteinases

The matrix metalloproteases (MMPs) are a multi-enzyme family of at least 24 members capable of completely degrading the components of the extracellular matrix (ECM), their natural substrates [26] [27] [28]. The ECM is a meshwork of cells and various types of collagens and proteoglycans, collectively called connective tissue, which provides mechanical support and helps to maintain the structural integrity of tissues and organs. The function of the ECM is particularly apparent in articular cartilage where it provides cushioning and ease of movement between bones in joints. The MMPs are secreted by the cellular components of the ECM (fibroblasts, chondrocytes and synoviocytes) and inflammatory cells (neutrophils and macrophages) in inactive forms (zymogens) which are converted extracellularly to the active enzymes by various proteinases. Normally, MMPs function in a highly regulated fashion as part of the physiological turnover of the ECM, effectively renewing and remodeling the ECM. However, in the clinical features of several diseases (including cancer), the ECM is degraded and there is much evidence to support that MMPs play a significant pathological role in ECM degradation [29].

At least twenty-four members of the MMP family have been identified and most assigned EC numbers; several more have been discovered recently. They can be classified generally into four subgroups based on substrate preference or cellular localization [30, 31]:

(a) collagenases (e.g., MMP-1, MMP-8, MMP-13), who preferentially degrade type I and type II collagen within the ECM

(b) gelatinases (e.g., MMP-2, MMP-9), who preferentially degrade type IV collagen and other proteins within the ECM and basal membrane

(c) stromelysins (e.g., MMP-3, MMP-10, MMP-11), who preferentially degrade proteoglycans within the ECM and proteolytically activate some serine proteases

(d) membrane type MMPs (MMP-14 to MMP-19), whose function is still under investigation. Membrane type MMPs are characterized by the presence of a hydrophobic transmembrane domain near the C-terminus for anchoring the protein in the cell membrane. All of the other MMPs are secreted into the extracellular milieu. Most of the known MMPs contain zinc in their catalytic sites and require calcium for activity. The major human MMPs have been cloned and exhibit greater than 50% homology. They contain a leader sequence for signaling their secretion by cells; a highly conserved pro-enzyme sequence removed upon activation; a catalytic site with a highly conserved zinc binding domain; and a carboxy terminal region containing a conserved sequence similar to hemopexin, a heme binding protein. Although MMPs can be readily activated in vitro using mercurial compounds or trypsin, the precise mechanism for propeptide removal and activation of MMPs in vivo is not understood. Some MMPs can undergo an autoactivation process, while recent evidence indicates that membrane type MMPs may function as activators of other MMPs.

A growing line of evidence implicates the MMPs act as important enzymes in cancer metastasis. Although different cancer cell lines have been shown to express various MMPs when grown in culture, gelatinase A in particular has been the focus of a number of recent studies which demonstrates its role in the invasiveness of cancer cells [32, 33]. For example, gelatinase A is found in the urine of bladder cancer patients, and specific monoclonal antibodies have been used to detect the enzyme in breast tumor sections [34]. The enzyme is expressed in an invasive prostate cancer cell line (PC-3 ML), and cells transfected with the gelatinase A gene are capable of extravasation when injected into mice [35]. These studies and others implicate the involvement of gelatinase A in tumor metastasis, and suggest that inhibitors of this enzyme—such as for example TIMP-3—may offer therapeutic potential in certain forms of cancer.

3.4 Metalloproteinases and Tissue Inhibitors of Metalloproteinases

It has been published that the activities of MMPs in normal tissues are thought to be regulated by the presence of TIMPs [9] [36]. The ratio of the amounts of TIMP and MMPs is thought to maintain a balance between the rates of degradation and synthesis of ECM. For example, in tissues from rheumatoid arthritics, an abnormally high expression of MMPs results in an imbalance of these enzymes and degradation of ECM [9, 37] [38].

Thus, the MMP/TIMP balance is important for ECM integrity; in cancer, this balance is shifted towards MMPs (through MMP expression by cancer cells) leading to the formation of new blood vessels (angiogenesis) and extravasation of tumor cells and metastasis in the end. The inventors successfully demonstrate that in vivo gene transfer induced expression of TIMP-3 can inhibit cancer growth—presumably through the combination of three effects:

(1) By shifting the MMP/TIMP balance in favor of TIMPs and thus preventing ECM degradation and metastasis (i.e., MMP-inhibitory activity).

(2) By inhibiting angiogenesis as it has recently been shown that TIMP-3 blocks the binding of VEGF to VEGF receptor-2 and inhibits downstream signaling and angiogenesis (i.e., antiangiogenic activity) [39]. This property seems to be independent of its MMP-inhibitory activity.

(3) By inducing apoptosis of cancer cells [18].

3.5 Cancer Treatment

The deficient activity of many antitumoral active compounds can be explained, at least to some degree, by the fact that the tumor cells within a tumor node are inaccessible to the antitumoral, in particular high molecular weight, active compounds [2-4]. Such active compounds have to diffuse through the vascular endothelium and the basal membrane, and through the tumor stroma and tumor parenchyma, in order to reach each individual tumor cell. The extent of this diffusion is essentially determined by the concentration or the concentration gradient of the active compound and its physicochemical characteristics. Moreover, convection, which is directed outwards by the higher pressure in the interior of the tumor, runs counter to the diffusion.

Since tumor blood vessels are accessible even to high molecular weight active compounds, it was consequently proposed at an early stage [40-43] to use tumor induced angiogenesis as target for anti-cancer therapy. Thus, attempts were made to inhibit tumor growth by using substances which inhibit angiogenesis [44]. Experimental investigations in animals demonstrated that systemic administration of substances which inhibit angiogenesis can also inhibit tumor proliferation. This applies, for example, to suramin [45], to heparin/steroid conjugates [46], to O-(chloroacetylcarbamoyl)fumigillol [47, 48], to monoclonal antibodies against angiogenin [49] and to angiostatin [50, 51]. However, these methods suffer from the disadvantages of systemic administration with peak concentrations of antiangiogenic factors shortly upon administration, their side effects and the risk of fresh tumor growth occurring once therapy is discontinued. In vivo gene transfer induced expression of TIMP-3—as demonstrated by the inventors—is able to overcome these limitations through the potential of local administration, continuous expression and thus continuous presence of the antiangiogenic factor, good safety profile and ongoing therapeutic effect.

Other anti-angiogenic strategies were developed with the purpose of inhibiting the blood supply of tumors by damaging endothelial cells so that the tumors necrose [42]. With this idea in mind, the administration was proposed of toxins, cytostatic agents or isotopes which were coupled to antibodies. These antibodies would be specific for the tumor-associated vascular endothelium. The intention was that the antibody conjugates would destroy the tumor-associated blood vessel in an endothelium-specific manner and thereby induce necrosis of the tumor [2]. Membrane antigens on the surface of endothelial cells were proposed as antigens for antibodies of this nature. These antigens include, for example, endoglin, endosialin, p96 dimer, VEGF receptors, PDGF receptors, urokinase (uPA) receptors and various adhesion molecules [2]. However, a feature possessed by all of these membrane antigens is that they are also present on non-proliferating endothelial cells, at least in relatively low concentrations. Since non-proliferating endothelial cells far outnumber proliferating endothelial cells even in a tumor-affected organism, this does not then adequately ensure the required tumor specificity of the effect which is sought by administering the antibody conjugate. Thus, also in this instance, in vivo gene transfer induced expression of TIMP-3—as demonstrated by the inventors—will prove a more useful and superior treatment method due to its lack of toxicity and potential for locally restricted action.

3.6 Gene Transfer

Gene transfer systems can be classified along different dimensions

(A) Nature or origin of the system

(B) Delivery mechanism

(C) Site of gene transfer (e.g., ex vivo, in vitro, in vivo)

(A) Based on the nature or origin of the gene transfer system, existing delivery systems for nucleic acid compositions can be subdivided into three groups: (1) viral vectors, (2) non-viral vectors, and (3) naked nucleic acids. Regarding vector targeting (specificity) and efficiency, viral vector systems are superior to conventional non-viral vectors and naked nucleic acids. On the other hand, non-viral vectors and naked nucleic acids are safer, easier to upscale in production and allow for the delivery of modified nucleic acids compared to viral vectors.

(B) Alternatively, based on the delivery mechanism, gene transfer methods fall into the following three broad categories: (1) physical (e.g., electroporation, direct gene transfer and particle bombardment), (2) chemical (e.g. lipid-based carriers and other non-viral vectors) and (3) biological (e.g. virus or bacterium derived vectors).

(C) Gene therapy methodologies can also be described by delivery site. Fundamental ways to deliver genes include ex vivo gene transfer, in vivo gene transfer, and in vitro gene transfer. In ex vivo gene transfer, cells are taken from the subject and grown in cell culture. The nucleic acid composition is introduced into the cells, the transduced or transfected cells are (in some instances) expanded in number and then reimplanted in the subject. In in vitro gene transfer, the transformed cells are cells growing in culture, such as tissue culture cells, and not particular cells from a particular subject. These “laboratory cells” are transfected or transduced; the transfected or transduced cells are then in some instances selected and/or expanded for either implantation into a subject or for other uses. In vivo gene transfer involves introducing the nucleic acid composition into the cells of the subject when the cells are within the subject.

Several delivery mechanisms may be used to achieve gene transfer in vivo, ex vivo, and/or in vitro.

Mechanical (i.e. physical) methods of DNA delivery can be achieved by direct injection of DNA, such as microinjection of DNA into germ or somatic cells, pneumatically delivered DNA-coated particles, such as the gold particles used in a “gene gun,” and inorganic chemical approaches such as calcium phosphate transfection. It has been found that physical injection of plasmid DNA into muscle cells yields a high percentage of cells which are transfected and have a sustained expression of marker genes. The plasmid DNA may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products. Particle-mediated gene transfer may also be employed for injecting DNA into cells, tissues and organs. With a particle bombardment device, or “gene gun,” a motive force is generated to accelerate DNA coated high density particles (such as gold or tungsten) to a high velocity that allows penetration of the target organs, tissues or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier mediated gene transfer through the use of fusogenic lipid vesicles such as liposomes or other vesicles for membrane fusion. A carrier harboring a DNA of interest can be conveniently introduced into body fluids or the bloodstream and then site specifically directed to the target organ or tissue in the body. Liposomes, for example, can be developed which are cell specific or organ specific. The foreign DNA carried by the liposome thus will be taken up by those specific cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer. Transfected DNA may also be complexed with other kinds of carriers so that the DNA is carried to the recipient cell and then resides in the cytoplasm or in the nucleoplasm of the recipient cell. DNA can be coupled to carrier nuclear proteins in specifically engineered vesicle complexes and carried directly into the nucleus. Carrier mediated gene transfer may also involve the use of lipid-based proteins which are not liposomes. For example, lipofectins and cytofectins are lipid-based positive ions that bind to negatively charged DNA, forming a complex that can ferry the DNA across a cell membrane. Another method of carrier mediated gene transfer involves receptor-based endocytosis. In this method, a ligand (specific to a cell surface receptor) is made to form a complex with a gene of interest and then injected into the bloodstream; target cells that have the cell surface receptor will specifically bind the ligand and transport the ligand-DNA complex into the cell.

Biological gene therapy methodologies usually employ viral vectors to insert genes into cells. The transduced cells may be cells derived from the patient's normal tissue, the patient's diseased tissue, or may be non-patient cells. Viral vectors that have been used for gene therapy protocols include but are not limited to, retroviruses, lentivruses, other RNA viruses such as poliovirus or Sindbis virus, adenovirus, adeno-associated virus, herpes viruses, simian virus 40, vaccinia and other DNA viruses.

Replication-defective murine retroviral vectors are commonly utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes flanked by 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most dividing cell types, precise single copy vector integration into target cell chromosomal DNA, and ease of manipulation of the retroviral genome. For example, altered retrovirus vectors have been used in ex vivo and in vitro methods to introduce genes into peripheral and tumor-infiltrating lymphocytes, hepatocytes, epidermal cells, myocytes, or other somatic cells (which may then be introduced into the patient to provide the gene product from the inserted DNA). For descriptions of various retroviral systems, see, e.g., U.S. Pat. No. 5,219,740; [52-56]. The main disadvantage of retroviral systems is that retroviral vectors can only infect dividing cells. This limitation is overcome by lentiviral vectors. Nevertheless, production of retro- and lentiviral vectors is complex, and the virions are not very stable compared to other viruses. More recently, the danger of inducing cancer through insertional mutagenesis has been raised as a major safety concern [57] [58].

A number of adenovirus based gene delivery systems have also been developed. Human adenoviruses are double stranded, linear DNA viruses with a protein capsid which enter cells by receptor-mediated endocytosis. Adenoviral vectors have a broad host range and are highly infectious, even at low virus titers. Moreover, adenoviral vectors can accommodate relatively long transgenes compared to other systems. A number of adenovirus based gene delivery systems have also been described [59-65]. The main limitation of adenoviral vectors is their high degree of immunogenicity, which limits their use in respect to applications that require long-term gene expression.

For many applications, long-term gene expression (over several years) will have to be achieved. This is also the case for the present invention. So far, primarily adeno-associated virus based vectors allow for this. Most other viral vectors are limited by expression of viral genes so that transduced cells will be eliminated by the immune system (e.g., adenoviral vectors), gene silencing (retroviral vectors or lentiviral vectors) or questionable safety profile (e.g., retroviral vectors or adenoviral vectors).

3.6.1 Adeno-Associated Viral Vectors

In its preferred embodiment, the present invention will use adeno-associated virus-based vectors [66] [67] [68] for the transfer of nucleic acid compositions into the appropriate target cells.

Adeno associated virus (AAV) is a small nonpathogenic virus of the parvoviridae family. AAV is distinct from the other members of this family by its dependence on a helper virus for replication. The approximately 5 kb genome of AAV consists of single stranded DNA of either plus or minus polarity. The ends of the genome are short inverted terminal repeats (ITRs) which can fold into hairpin structures and serve as the origin of viral DNA replication. Physically, the parvovirus virion is non-enveloped and its icosohedral capsid is approximately 20 nm in diameter. To date, at least 8 serologically distinct AAVs have been identified and isolated from humans or primates and are referred to as AAV types 1-8. The most extensively studied of these isolates are AAV type 2 (AAV2) and AAV type 5 (AAV5).

The genome of AAV2 is 4680 nucleotides in length and contains two open reading frames (ORFs). The left ORF encodes the non-structural Rep proteins, Rep40, Rep52, Rep68 and Rep78, which are involved in regulation of replication and transcription in addition to the production of single-stranded progeny genomes. Furthermore, two of the Rep proteins have been associated with the preferential integration of AAV2 genomes into a region of the q arm of human chromosome 19. Rep68/78 have also been shown to possess NTP binding activity as well as DNA and RNA helicase activities. The Rep proteins possess a nuclear localization signal as well as several potential phosphorylation sites. Mutation of one of these kinase sites resulted in a loss of replication activity. The ends of the genome are short inverted terminal repeats which have the potential to fold into T-shaped hairpin structures that serve as the origin of viral DNA replication. Within the ITR region two elements have been described which are central to the function of the ITR, a GAGC repeat motif and the terminal resolution site (trs). The repeat motif has been shown to bind Rep when the ITR is in either a linear or hairpin conformation. This binding serves to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The right ORF of AAV2 encodes related capsid proteins referred to as VP1, 2 and 3. These capsid proteins form the icosahedral, non-enveloped virion particle of ˜20 nm diameter. VP1, 2 and 3 are found in a ratio of 1:1:10. The capsid proteins differ from each other by the use of alternative splicing and an unusual start codon. Deletion analysis has shown that removal or alteration of VP1, which is translated from an alternatively spliced message, results in a reduced yield of infectious particles. Mutations within the VP3 coding region result in the failure to produce any single-stranded progeny DNA or infectious particles.

The findings described in the context of AAV2 are generally applicable to other AAV serotypes as well. The following features of AAV have made it an attractive vector for gene transfer. AAV vectors possess a broad host range [69], transduce both dividing and non dividing cells in vitro and in vivo and maintain high levels of expression of the transduced genes in the absence of a significant immune response to the transgene product in general. Moreover, as wild-type AAV is non-pathogenic, AAV vector particles are assumed to be non-pathogenic as well (in contrast to adenoviral vectors). Viral particles are heat stable, resistant to solvents, detergents, changes in pH and temperature. The ITRs have been shown to be the only cis elements required for replication and packaging and may contain some promoter activities. Thus, no viral genes are encoded by AAV vectors.

Vectors based on adeno-associated virus (AAV) emerged as those preferred for achieving truly stable transduction following in vivo administration. Although the stability of transgene expression with AAV2-based vectors is impressive, its efficiency often is not. Isolation and characterization of several other AAV serotypes provided new opportunities for vector development. For example, Chiorini and colleagues created replication defective versions of AAV serotype 5 (AAV5) for gene transfer [70]. Transduction efficiency was substantially improved with AAV5-based vectors when compared with those based on AAV2 in several applications, including those involving muscle and lung. Another improvement in the art was the creation of hybrid vectors based on AAV2 inverted terminal repeats (ITRs) produced with AAV2 rep and AAV5 cap [71]. The resulting defective vector packages an AAV2 genome in an AAV5 capsid. The transduction efficiency of the AAV2/5 hybrid is superior to that of AAV2 in lung, muscle, and retina. A further advantage of AAV vectors based on serotype 5 capsids is that humans do not harbor antibodies capable of interfering with AAV5 transduction.

### SUMMARY OF THE INVENTION

Cancer is a major cause of death in developed countries. Yet, there is still a high degree of unmet need in the prevention, treatment and/or cure of cancer. The present invention relates to methods for treating cancer, preventing cancer and/or inhibiting the growth of cancer (cells) by administering to a mammalian subject a gene transfer vector in vivo comprising a nucleic acid composition whose expression directly or indirectly leads to the expression and/or secretion of Tissue inhibitor of metalloproteinase-3 (TIMP-3). Upon successful transduction, expression and/or secretion of TIMP-3—either locally (in the vicinity or within the cancer cells) or systemically—will inhibit cancer growth. Also provided are pharmaceutical kits containing the gene transfer vector in a suitable pharmaceutical suspension for administration.

In one aspect, the invention provides a method for preventing, treating and/or inhibiting progression of cancer by local and/or systemic expression and/or secretion of TIMP-3. The method involves administering to a mammalian subject in vivo a gene transfer vector comprising a transgene whose expression directly or indirectly leads to the expression and/or secretion of TIMP-3.

In another aspect, the invention provides a pharmaceutical kit for delivery of said gene transfer vector. The kit may contain a container for administration of a predetermined dose. The kit further may contain a suspension containing the gene transfer vector for delivery of a predetermined dose, said suspension comprising

(a) the gene transfer vector comprising a nucleic acid composition comprising a transgene whose expression directly or indirectly leads to the expression and/or secretion of TIMP-3; and

(b) a physiologically compatible carrier.

The inventors are the first to demonstrate the anti-cancer effect of in vivo gene transfer induced expression of TIMP-3. In vivo gene transfer induced expression of TIMP-3 offers advantages over existing treatments:

The deficient activity of many antitumoral active compounds can be explained, at least to some degree, by the fact that the tumor cells within a tumor node are inaccessible to the antitumoral, in particular high molecular weight, active compounds [2-4]. Moreover, many tumor cells develop resistance to current treatment modalities. In vivo gene transfer induced expression of TIMP-3 overcomes these limitations as its prime target of action are not the tumor cells themselves, but the surrounding (non-cancerous) tissue and the inhibition of ECM degradation and neovascularization of tumors (angiogenesis). As ECM degradation and angiogenesis plays a crucial role in the survival of tumors, interference with these processes through expression and secretion of TIMP-3 results in the inhibition of cancer growth—as demonstrated by the inventors.

In addition, TIMP-3 combines three anti-cancer effects: Antiangiogenic activity (and thus inhibition of neovascularization), inhibition of MMPs (and thus ECM degradation and metastasis), and apoptotic activity (and thus inducing apoptosis of cancer cells)—an advantage over other treatment methods currently in development that address inhibition of MMPs, angiogenesis and apoptosis separately. Besides, compared to other antiangiogenic therapies such as small molecules or protein therapies, the in vivo gene transfer-induced expression of TIMP-3 guarantees both a high local concentration of the antiangiogenic factor close to the tumor and continuous presence of the antiangiogenic factor due to constitutive synthesis.

Last, given the alternative treatment paradigm of TIMP-3 compared to e.g., cytotoxic drugs or radiation therapy, in vivo gene transfer induced expression of TIMP-3 might function in addition to current treatments to increase overall treatment efficacy.

Other aspects and advantages of the invention will be readily apparent to one of skill in the art from the detailed description of the invention.

### DETAILED DESCRIPTION OF THE INVENTION

The present invention relates to methods for treating cancer, preventing cancer and/or inhibiting the growth of cancer (cells) by administering to a mammalian subject in vivo a gene transfer vector comprising a nucleic acid composition whose expression directly or indirectly leads to the expression and/or secretion of Tissue Inhibitor of metalloproteinase-3 (TIMP-3). Upon successful transduction, expression and secretion of TIMP-3—either locally (in the vicinity or within the cancer cells) or systemically—will inhibit cancer growth. Also provided are pharmaceutical kits containing the gene transfer vector in a suitable pharmaceutical suspension for administration.

The practice of the present invention will employ, unless otherwise indicated, conventional methods of virology, microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

It must be noted that as used herein and in the appended claims, the singular forms “a” and “the” include plural referents unless the context clearly dictates otherwise. Thus, for example, reference to “a cell” or “the cell” includes a plurality (“cells” or “the cells”), and so forth. Moreover, the word “or” can either be exclusive in nature (i.e., either A or B, but not A and B together), or inclusive in nature (A or B, including A alone, B alone, but also A and B together). One of skill in the art will realize which interpretation is the most appropriate—unless it is detailed by reference in the text as “either A or B” (exclusive “or”) or “and/or” (inclusive “or”).

### 1 DEFINITIONS

In describing the present invention, the following terms will be employed, and are intended to be defined as indicated below.

For purposes of this invention, by “Tissue inhibitor of metalloproteinase-3” or “TIMP-3” is meant a protein identical and/or substantially homologous to the following amino acid sequence (NCBI accession number CAA53813):

| | | | | | | |
|---|---|---|---|---|---|---|
| 1 | mtpwlglivl | lgswslgdwg | aeactcspsh | pqdafcnsdi | virakvvgkk | lvkegpfgtl |
| 61 | vytikqmkmy | rgftkmphvq | yihteasesl | cglklevnky | qylltgrvyd | gkmytglcnf |
| 121 | verwdqltls | qrkglnyryh | lgcnckiksc | yylpcfvtsk | neclwtdmls | nfgypgyqsk |
| 181 | hyacirqkgg | ycswyrgwap | pdksiinatd | p | | |

Human TIMP-3 is encoded by the following genetic sequence (NCBI accession number X76227):

| | | | | | | |
|---|---|---|---|---|---|---|
| 1 | atgacccctt | ggctcgggct | catcgtgctc | ctgggcagct | ggagcctggg | ggactggggc |
| 61 | gccgaggcgt | gcacatgctc | gcccagccac | ccccaggacg | ccttctgcaa | ctccgacatc |
| 121 | gtgatccggg | ccaaggtggt | ggggaagaag | ctggtaaagg | aggggccctt | cggcacgctg |
| 181 | gtctacacca | tcaagcagat | gaagatgtac | cgaggcttca | ccaagatgcc | ccatgtgcag |
| 241 | tacatccaca | cggaagcttc | cgagagtctc | tgtggcctta | agctggaggt | caacaagtac |
| 301 | cagtacctgc | tgacaggtcg | cgtctatgat | ggcaagatgt | acacggggct | gtgcaacttc |
| 361 | gtggagaggt | gggaccagct | caccctctcc | cagcgcaagg | ggctgaacta | tcggtatcac |
| 421 | ctgggttgta | actgcaagat | caagtcctgc | tactacctgc | cttgctttgt | gacttccaag |
| 481 | aacgagtgtc | tctggaccga | catgctctcc | aatttcggtt | accctggcta | ccagtccaaa |
| 541 | cactacgcct | gcatccggca | gaagggcggc | tactgcagct | ggtaccgagg | atgggccccc |
| 601 | ccggataaaa | gcatcatcaa | tgccacagac | ccctga | | |

One of ordinary skill in the art can make changes to the displayed genetic sequence without changing the amino acid sequence of the resulting translation product.

For purpose of this invention, the term “TIMP-3 inducing minigene” refers to a minigene comprising a nucleic acid composition whose expression directly or indirectly leads to the expression and/or secretion of TIMP-3 or a substantially homologous protein in the transduced host cell upon gene transfer. In one embodiment, the TIMP-3 inducing minigene comprises the human TIMP-3 coding sequence (NCBI accession number X76227); in another embodiment, the TIMP-3 inducing minigene comprises the coding sequence for a transcription factor whose expression leads to the expression of therapeutic amounts of TIMP-3 alone or in combination with the expression of other external or internal transcription factors. In yet another embodiment, the TIMP-3 inducing minigene comprises only a part of the TIMP-3 coding sequence. The target cell is then transduced with two TIMP-3 inducing minigenes—each encoding one part of the TIMP-3 coding sequence. Both minigenes combined then will lead to the expression and secretion of TIMP-3. Such an approach is feasible for example by using AAV trans-splicing vectors. How to create such AAV trans-splicing vectors has been described in the art [72-74]. In yet another embodiment, the TIMP-3 inducing minigene comprises only a part of a coding sequence for a specific transcription factor. The target cell is then transduced with two TIMP-3 inducing minigenes—each encoding one part of the coding sequence of said specific transcription factor. Both minigenes combined then will lead to the expression and secretion of TIMP-3. Such an approach is feasible for example by using AAV trans-splicing vectors. How to create such AAV trans-splicing vectors has been described in the afore mentioned prior art. This invention claims all theoretically possible nucleic acid compositions of TIMP-3 inducing minigenes whose transduction of host cells will lead either indirectly or directly to the expression and/or synthesis and/or secretion of TIMP-3. Generally, a minigene may have a size in the range of several hundred base pairs up to about 30 kb.

For purposes of this invention, the term “gene therapy” means the transfer of nucleic acid compositions into cells of a multicellular eukaryotic organism, be it in vivo, ex vivo or in vitro (see also [75] [76]). The term “gene therapy” should not be limited to the purpose of correcting metabolic disorders, but be interpreted more as a technical term for the transfer of nucleic acid compositions for therapeutic purposes in general, independent of a specific therapeutic purpose. Therefore, the term “gene therapy” would include—without limitation—correction of metabolic disorders, cancer therapy, vaccination, monitoring of cell populations, cell expansion, stem cell manipulation etc. by means of transfer of nucleic acid compositions.

For purposes of this invention, “transfection” is used to refer to the uptake of nucleic acid compositions by a cell. A cell has been “transfected” when an exogenous nucleic acid composition has crossed the cell membrane. A number of transfection techniques are generally known in the art. See, e.g., [77, 78], Sambrook et al. (1989) Molecular Cloning, a laboratory manual, Cold Spring Harbor Laboratories, New York, Davis et al. (1986) Basic Methods in Molecular Biology, Elsevier, and [79]. Such techniques can be used to introduce one or more nucleic acid compositions, such as a plasmid vector and other nucleic acid molecules, into suitable host cells. The term refers to both stable and transient uptake of the genetic material. For purposes of this invention, “transduction” is a special form of “transfection” via a viral vector.

For purposes of this invention, “transduction” denotes the delivery of a nucleic acid composition to, into or within a recipient cell either in vivo, in vitro or ex vivo, via a virus or viral vector, such as via a recombinant AAV virion. Transduction is a special form of transfection, i.e., the term transfection includes the term transduction.

For purposes of this invention, “nucleic acid composition transfer”, “nucleic acid composition delivery”, “gene transfer” or “gene delivery” refers to methods or systems for transferring nucleic acid compositions into host cells. Such methods can result in transient expression of non-integrated transferred DNA, extrachromosomal replication and expression of transferred replicons (e.g., episomes), or integration of transferred genetic material into the genomic DNA of host cells. Nucleic acid composition transfer provides a unique approach for the treatment of inherited and acquired diseases including cancer. A number of systems and methods have been developed for nucleic acids composition transfer into mammalian cells.

For purposes of this invention, by “vector”, “transfer vector”, “gene transfer vector” or “nucleic acid composition transfer vector” is meant any element, such as a plasmid, phage, transposon, cosmid, chromosome, virus, virion, etc., which is capable of transferring and/or transporting a nucleic acid composition to a host cell, into a host cell and/or to a specific location and/or compartment within a host cell. Thus, the term includes cloning and expression vehicles, as well as viral and non-viral vectors and potentially naked or complexed DNA. However, the term does not include cells that produce gene transfer vectors such as retroviral packaging cell lines.

For purposes of this invention, by “AAV vector”, “AAV-based vector”, “adeno-associated virus based vector” or “adeno-associated viral vector” is meant a vector derived from an adeno-associated virus serotype, including without limitation, AAV-1, AAV-2, AAV-3, AAV-4, AAV-5, AAV-6, AAV-7, AAV-8 etc. or any other virus or serotype which is substantially homologous in its capsid protein sequence to the AAV5 capsid protein sequence. The term also includes hybrid vectors combining characteristics of more than one AAV serotype. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging.

For purposes of this invention, by “recombinant virus”, “recombinant virion”, “recombinant vector” or “recombinant viral vector” is meant a virus that has been genetically altered, e.g., by the addition or insertion of a heterologous nucleic acid composition into the particle.

For purposes of this invention, by “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion, and both strands are equally infectious.

For purposes of this invention, a “recombinant AAV virion” or “rAAV virion” is defined herein as an infectious, replication-defective virus composed of an AAV protein shell, encapsidating a heterologous DNA molecule of interest which is flanked on one or both sides by AAV ITRs. A rAAV virion is produced in a suitable host cell which has had an AAV vector, AAV helper functions and accessory functions introduced therein. In this manner, the host cell is rendered capable of encoding AAV polypeptides that are required for packaging the AAV vector (comprising a recombinant nucleotide sequence of interest) into recombinant virion particles for subsequent gene delivery. The term “rAAV virion” and its synonyms and the term “AAV vector” and its synonyms can be used interchangeably.

For purposes of this invention, “pseudotyped” (r)AAV refers to a recombinant AAV in which the capsid protein is of a serotype heterologous to the serotype(s) of the ITRs of the minigene. For example, a pseudotyped rAAV may be composed of a minigene carrying AAV5 ITRs and capsid of AAV2, AAV1, AAV3, AAV4, AAV6, AAV7, AAV8 or another suitable AAV serotype, where the minigene is packaged in the heterologous capsid. Alternatively, a pseudotyped rAAV may be composed of an AAV5 capsid which has packaged therein a minigene containing ITRs from at least one of the other serotypes. Particularly desirable rAAV composed of AAV5 are described in U.S. patent application Ser. No. 60/200,409, filed Apr. 28, 2000 and International Patent Application No. PCT/USO1/13000, filed Apr. 23, 2001, both of which are incorporated by reference herein.

As defined herein, AAV capsid proteins include hybrid capsid proteins which contain a functional portion of one or more AAV capsid proteins. Such hybrid capsid proteins may be constructed such that a fragment of a capsid derived from one serotype is fused to a fragment of a capsid from another serotype to form a single hybrid capsid which is useful for packaging of an AAV minigene.

For purposes of this invention, the term “protein” means a polypeptide (native (i.e., naturally-occurring) or mutant), oligopeptide, peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

For purpose of this invention, “desired protein” refers to proteins encoded by minicassettes or minigenes used in the present invention, which either act as target proteins for an immune response, or as a therapeutic or compensating protein in gene therapy regimens.

For purposes of this invention, by “DNA” is meant a polymeric form of desoxyribonucleotides (adenine, guanine, thymine, or cytosine) in double-stranded or single-stranded form, either relaxed and supercoiled, either linear or circular. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The term captures molecules that include the four bases adenine, guanine, thymine, or cytosine, as well as molecules that include base analogues which are known in the art.

For purposes of this invention, “polynucleotide” as used herein means a polymeric form of nucleotides of any length, either ribonucleotides or desoxyribonucleotides. This term refers only to the primary structure of the molecule. Thus, the term includes double- and single-stranded DNA, as well as, double- and single-stranded RNA. It also includes modifications, such as methylation or capping, and unmodified forms of the polynucleotide.

For purposes of this invention, the term “nucleic acid composition” means any nucleic acid molecule, may it be single stranded, double stranded or triple helical or a mixture thereof, may it be DNA, RNA, PNA, a DNA/RNA hybrid (e.g., a chimeraplast), may it be linear or circular, chemically modified, coupled to other macromolecules (e.g. proteins) or a mixture thereof, independent of its size (single nucleotide, oligonucleotide, polynucleotide). It may be in the form of a plasmid, cosmid, bacteriophage-based genome (e.g., M13-based vectors), viral vector such as an adenoviral genome, adeno-associated viral genome, retroviral genome, lentiviral genome, herpes virus genome, bacterial artificial chromosome, yeast artificial chromosome, mammalian artificial chromosome, or any part or parts or combinations thereof. The nucleic acid composition comprises a nucleotide sequence that encodes a desired protein or peptide, serves as a template for functional nucleic acid molecules and/or functions as a functional unit in itself such as—without limitation—a ribozyme, an antisense molecule or an aptamer. The desired protein/peptide and/or functional nucleic acid molecule may be any product of medical, industrial or scientific interest. In many instances, the nucleic acid composition functions as a “transgene”. In the context of this invention, the nucleic acid composition by itself or by its encoded product leads either directly or indirectly to the expression and secretion of TIMP-3. In one specific embodiment, this is achieved by selecting TIMP-3 as transgene. In another embodiment, the transgene is a transcription factor protein that induces the transcription of TIMP-3 in cells, where said gene typically is not expressed (or not expressed in therapeutic levels) in said specific type of cells. The transcription factor protein might either be a naturally occurring transcription factor that is normally not or not sufficiently expressed in the cells of interest, or it might be a synthetic transcription factor protein. How to create synthetic transcription factors (e.g., based on the zinc finger motif), has been published, and those skilled in the art can create zinc fingers to activate any given gene without undue experimentation [80-84].

The nucleic acid composition can perform different functions in the context of this invention: The most important embodiments for the present invention include the local and/or systemic expression and secretion of TIMP-3 to treat/prevent hyperproliferative diseases (cancer). The expression, secretion and/or functionality of TIMP-3 might be regulated. One of skill in the art can generate any configuration of nucleic acid compositions, regulation mechanisms, and transfer vectors which can be used via the methods described herein to achieve expression and secretion of TIMP-3 in therapeutic levels (Sambrook 1989, Lodish et al. 2000). According to the definition used herein, all these applications would be classified as gene therapy.

For the purpose of describing the relative position of nucleotide sequences in a particular nucleic acid molecule throughout the instant application, such as when a particular nucleotide sequence is described as being situated “upstream,” “downstream,” “5′,” or “3”′ relative to another sequence, it is to be understood that it is the position of the sequences in the non-transcribed strand of a DNA molecule that is being referred to as is conventional in the art.

For purposes of this invention, a “gene sequence” or “coding sequence” or “protein coding sequence” or “open reading frame” or a sequence which “encodes” a particular protein, is a nucleic acid composition which is transcribed into RNA (in the case of DNA) and potentially translated (in the case of mRNA) into a polypeptide in vitro or in vivo when placed under the control of appropriate regulatory sequences. The boundaries of the gene are determined by a start codon at the 5′ (amino) terminus and potentially a translation stop codon at the 3′ (carboxy) terminus. A gene sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the protein coding sequence.

For purposes of this invention, by the term “transgene” is meant a nucleic acid composition made out of DNA, which encodes a peptide, oligopeptide or protein. The transgene may be operatively linked to regulatory components in a manner which permits transgene transcription, translation and/or ultimately directs expression of a product encoded by the nucleic acid composition in the host cell, e.g., the transgene is placed into operative association with a promoter and enhancer elements, as well as other regulatory sequences, such as introns or polyA sequences, useful for its regulation. The composite association of the transgene with its regulatory sequences is referred to herein as a “minicassette” or “minigene”. Minicassettes or minigenes in their entirety are also nucleic acid compositions. The exact nucleic acid composition will depend upon the use to which the resulting nucleic acid transfer vector will be put and is known to the artisan (Sambrook 1989, Lodish et al. 2000). When taken up by a target cell, the nucleic acid composition may remain present in the cell as a functioning extrachromosomal molecule, or it may integrate into the cell's chromosomal DNA, depending on the kind of transfer vector used.

For purposes of this invention, “AAV minigene” refers to a construct composed of, at a minimum, AAV ITRs and a heterologous nucleic acid composition. For production of rAAV according to the invention, a minigene may be carried on any suitable vector, including viral vectors, plasmid vectors, and the like.

For purposes of this invention, “heterologous” as it relates to nucleic acid compositions denotes sequences that are not normally joined together. Thus, a “heterologous” region of a nucleic acid composition is a segment of nucleic acid within or attached to another nucleic acid composition that is not found in association with the other molecule in nature. For example, a heterologous region of a nucleic acid composition could include a coding sequence flanked by sequences not found in association with the coding sequence in nature. Another example of a heterologous coding sequence is a construct where the coding sequence itself is not found in nature (e.g., synthetic sequences having codons different from the native gene). Allelic variation or naturally occurring mutational events do not give rise to heterologous DNA, as used herein.

For purposes of this invention, the term “control elements” or “regulatory sequences” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present as long as the selected coding sequence is capable of being replicated, transcribed and/or translated in an appropriate host cell. Sometimes, the entirety of control elements and coding sequence is referred to as “gene”; in other instances, “gene” only refers to the coding sequence. For purposes of this invention, “gene” refers to the entirety of control elements and coding sequence. Expression control elements include appropriate transcription initiation, termination, promoter and enhancer sequences, efficient RNA processing signals such as splicing and polyadenylation signals, sequences that stabilize cytoplasmic mRNA, sequences that enhance translation efficacy (i.e., Kozak consensus sequence), sequences that enhance protein stability, and when desired, sequences that enhance protein processing and/or secretion. A great number of expression control elements, e.g., native, constitutive, inducible and/or tissue specific, are known in the art and may be utilized to drive expression of the gene, depending upon the type of expression desired. For eukaryotic cells, expression control elements typically include a promoter, an enhancer, such as one derived from an immunoglobulin gene, SV40, cytomegalovirus, etc., a polyadenylation sequence, and may include splice donor and acceptor sites. The polyadenylation sequence generally is inserted following the transgene sequences and before the 3′ ITR sequence in rAAV vectors. In one embodiment, the bovine growth hormone polyA is used.

The regulatory sequences useful in the constructs of the present invention may also contain an intron, desirably located between the promoter/enhancer sequence and the gene. One possible intron sequence is derived from SV40, and is referred to as the SV40 T intron sequence. Another suitable regulatory sequence includes the woodchuck hepatitis virus post-transcriptional element [85]. Still other methods may involve the use of a second internal promoter, an alternative splice signal, a co- or post-translational proteolytic cleavage strategy, among others which are known to those of skill in the art. Selection of these and other common vector and regulatory sequences are conventional, and many such sequences are available. See, e.g., Sambrook et al, and references cited therein at, for example, pages 3.18-3.26 and 16.17-16.27 and Ausubel et al., CURRENT PROTOCOLS IN MOLECULAR BIOLOGY, John Wiley & Sons, New York, 1989.

One of skill in the art may make a selection among these regulatory sequences without departing from the scope of this invention. Suitable promoter/enhancer sequences may be selected by one of skill in the art using the guidance provided by this application. Such selection is a routine matter and is not a limitation of the present invention. For instance, one may select one or more regulatory sequences operably linked to the TIMP-3 coding sequence as expression cassette for insertion in a “AAV minigene” which is composed of the 5′ ITRs, the TIMP-3 expression cassette, and 3′ ITRs in the context of rAAV vectors. Thus, this system permits a great deal of latitude in the selection of the various components of the minigene. Provided with the teachings of this invention, the design of such a minigene can be made by resort to conventional techniques.

For purposes of this invention, the term “promoter” means a regulatory sequence capable of binding RNA polymerase and/or a regulatory sequence sufficient to direct transcription. “Promoter” is also meant to encompass those promoter (or enhancer) elements for cell-type specific, tissue-specific and/or inducible (by external signals or agents) transcription; such elements may be located in the 5′ or 3′ regions of a native gene.

In one embodiment, lung-specific promoters are desired. Examples of such lung-specific promoters include Clara cell secretory protein (CCSP) promoter [86]; the lung-specific surfactant protein C promoter [87]; Jaagskiekte sheep retrovirus (JSRV) long terminal repeat [88]; rat aquaporin-5 promoter [89]. Still other lung-specific promoters may be readily selected by one of skill in the art for use in the invention. Alternatively, non-tissue-specific promoters may be readily selected.

In another embodiment, high-level constitutive expression is desired. Examples of such promoters include, without limitation, the retroviral Rous sarcoma virus (RSV) LTR promoter (optionally with the RSV enhancer), the cytomegalovirus (CMV) promoter (optionally with the CMV enhancer) [90], the SV40 promoter, the dihydrofolate reductase promoter, the .beta.-actin promoter, the phosphoglycerol kinase (PGK) promoter, and the EF1.alpha. promoter (Invitrogen).

Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter [91], the tetracycline-repressible system [92], the tetracycline-inducible system [93]; see also U.S. patent application 0030013189, [94], the RU486-inducible system [95, 96] and the rapamycin-inducible system [97]. Other types of inducible promoters which may be useful in the transgenes and other constructs described herein are those which are regulated by a specific physiological state, e.g., temperature, acute phase, a particular differentiation state of the cell, or in replicating cells only.

For purposes of this invention, the term “operative association” or “operative linkage” refers to an arrangement of elements or nucleic acid sequences wherein the components so described are configured so as to perform their intended function. Thus, (a) regulatory sequence(s) operably linked to a coding sequence are capable of effecting the expression of said coding sequence and are connected in such a way as to permit gene expression of the coding sequence when the appropriate molecules (e.g., transcriptional activator proteins) are bound to the regulatory sequence(s). The regulatory sequences need not be contiguous with the coding sequence, as long as they function to direct the expression thereof. Thus, for example, intervening untranslated yet transcribed sequences can be present between a promoter sequence and the coding sequence and the promoter sequence can still be considered “operably linked” to the coding sequence. “Operably linked” sequences include both expression control sequences that are contiguous with the coding sequences for the product of interest and expression control sequences that act in trans or at a distance to control the expression of the product of interest.

For purposes of this invention, “homology” or “homologous” refers to the percent homology between two polynucleotide or two polypeptide moiety. The correspondence between the sequence from one moiety to another can be determined by techniques known in the art. Two DNA or two polypeptide sequences are “substantially homologous” to each other when at least about 80%, preferably at least about 90%, and most preferably at least about 95% of the nucleotides or amino acids match over a defined length of the molecules, as determined using methods in the art.

The techniques for determining amino acid sequence homology are well-known in the art. In general, “homology” means the exact amino acid to amino acid comparison of two or more polypeptides at the appropriate place, where amino acids are identical or possess similar chemical and/or physical properties such as charge or hydrophobicity. A so-termed “percent homology” then can be determined between the compared polypeptide sequences. The programs available in the Wisconsin Sequence Analysis Package (available from Genetics Computer Group, Madison, Wis.), for example, the GAP program, are capable of calculating homologies between two polypeptide sequences. Other programs for determining homology between polypeptide sequences are known in the art.

Homology for polynucleotides is determined essentially as follows: Two polynucleotides, one of which is the full length TIMP-3 cDNA sequence (NCBI accession number X76227), are considered to be “substantially homologous” to each other when at least about 80%, preferably at least about 90%, and most preferably at least about 95% of the nucleotides match over a defined length of the molecules, when aligned using the default parameters of the search algorithm BLAST 2.0. The BLAST 2.0 program is publicly available.

Alternatively, homology for polynucleotides can be determined by hybridization experiments. As used herein, a nucleic acid sequence or fragment (such as for example, primers or probes), is considered to selectively hybridize to a TIMP-3 sequence, thus indicating “substantial homology”, if such a sequence is capable of specifically hybridizing to the TIMP-3 sequence or a variant thereof (e.g. a probe that hybridizes to TIMP-3 nucleic acid but not to a polynucleotide from other members of the TIMP family) or specifically priming a polymerase chain reaction: (i) under typical hybridization and wash conditions, such as those described, for example, in Maniatis, (Molecular Cloning: A Laboratory Manual, 2^{nd} Edition, 1989) where preferred hybridization conditions are those of lesser stringency and more preferred, higher stringency; or (ii) using reduced stringency wash conditions that allow at most about 25-30% basepair mismatches, for example, 2.times.SSC, 0.1% SDS, at room temperature twice, for 30 minutes each; then 2.times.SSC, 0.1% SDS, 37 C, once for 30 minutes; the 2.times.SSC at room temperature twice, 10 minutes each or (iii) under standard PCR conditions or under “touch-down” PCR conditions such as described by [98]).

For purposes of this invention, the term “cell” means any prokaryotic or eukaryotic cell, either ex vivo, in vitro or in vivo, either separate (in suspension) or as part of a higher structure such as—but not limited to—organs or tissues.

For purposes of this invention, “lung cells” may refer to one or more of the following types of cells (without limitation): type I pneumocytes, type II pneumocytes, pseudostratified columnar epithelial cells, stratified squamous epithelial cells, gland cells, duct cells, subepithelial connective tissue cells, goblet cells, mucosal cells, submucosal cells, hyaline cartilage cells, perichondrial cells, ciliated columnar cells, basal epithelial cells, brush cells, bronchial epithelial cells, submucosal gland cells, pseudostratified ciliated columnar epithelial cells, lung tissue cells, bronchial respiratory epithelial cells, cuboid epithelial cells of brionchioles, bronchiolar epithelial cells, alveolar cells, squamous (type 1) alveolar cells, great (type II) alveolar cells, and alveolar macrophages.

For purposes of this invention, the term “host cell” means a cell that can be transduced and/or transfected by an appropriate gene transfer vector. The nature of the host cell may vary from gene transfer vector to gene transfer vector. In more specific contexts, “host cell” refers to a cell that allows for production of recombinant viral vectors. In one specific embodiment of this invention, the host cell is the human embryonic kidney (HEK) cell line 293 for the production of rAAV virions. In that specific context, the term “packaging cell” or “packaging cell line” is used as a synonym for “host cell”.

For purposes of this invention, by the term “a Therapeutic of this invention” is meant any gene transfer vector that allows for direct or indirect expression and/or secretion of the TIMP-3 protein or a substantially homologous protein by means of in vivo gene transfer.

For purposes of this invention, “treatment” refers to prophylaxis and/or therapy. “Pharmaceutically effective” levels are levels sufficient to achieve a physiologic effect in a human or veterinary subject, which effect may be therapeutic or prophylactic.

For purposes of this invention, by “mammalian subject” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

For purposes of this invention, the terms “individual” or “subject” or “patient” as used herein refer to vertebrates, particularly members of the mammalian species and include but are not limited to domestic animals, sports animals, primates and humans; more particularly the term refer to humans.

### 2 GENERAL METHODS

The present invention provides for the successful in vivo transfer of a gene transfer vector comprising a nucleic acid composition encoding TIMP-3 or (an)other gene(s) whose expression result(s) in the subsequent expression of TIMP-3. In its preferred embodiment, the method allows for the direct, in vivo administration of recombinant AAV virions. One of skill in the art will be able to use alternative gene transfer vectors such as—without limitation—retroviral vectors, lentiviral vectors, adenoviral vectors, Herpes virus based vectors etc., which would still be within the scope of this invention.

The practice of the present invention will employ, unless otherwise indicated, conventional methods of virology, microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

2.1 Recombinant AAV Virions

The recombinant AAV virions of the preferred embodiment, comprising a TIMP-3 inducing minigene, can be produced using standard methodology, known to the artisan. The methods generally involve the steps of

(1) introducing an AAV vector construct into a host cell (e.g., 293 cells);

(2) introducing an AAV packaging construct into the host cell, where the packaging construct includes AAV coding regions (e.g., rep and cap sequences) capable of being expressed in the host cell to complement AAV packaging functions missing from the AAV vector construct;

(3) introducing one or more helper viruses and/or accessory function vector constructs into the host cell, wherein the helper virus and/or accessory function vector constructs provide accessory functions capable of supporting efficient recombinant AAV (“rAAV”) virion production in the host cell; and

(4) culturing the host cell to produce rAAV virions.

The AAV vector construct, AAV packaging construct and the helper virus or accessory function vector constructs can be introduced into the host cell either simultaneously or serially, using standard transfection techniques.

In one embodiment, pseudotyped rAAV is produced, in which a non-AAV5 serotype ITR based TIMP-3 inducing minigene is packaged in an AAV5 capsid. The inventors have previously found that this pseudotyping can be achieved by utilizing a Rep protein (or a functional portion thereof) of the same serotype or a cross-reactive serotype as that of the ITRs found in the minigene in the presence of sufficient packaging and accessory functions to permit packaging [71]. Thus, an AAV2 minigene (harboring a TIMP-3-inducing expression cassette) can be pseudotyped in an AAV5 capsid by use of a rep protein from AAV2 or a cross-reactive serotype, e.g., AAV1, AAV3, AAV4 or AAV6. Similarly, an AAV minigene containing AAV1 5′ ITRs and AAV2 3′ ITRs may be pseudotyped in an AAV5 capsid by use of a Rep protein from AAV 1, AAV2, or another cross-reactive serotype. However, because AAV5 is not cross-reactive with the other AAV serotypes, an AAV5 minigene can be pseudotyped in a heterologous AAV capsid only by use of an AAV5 Rep protein.

he host cell for rAAV virion production itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, W138, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirements for the cell used is that it not carry any adenovirus gene other than E1, E2a and/or E4 ORF6; it not contain any other virus gene which could result in homologous recombination of a contaminating virus during the production of rAAV; and it is capable of infection or transfection of DNA and expression of the transfected DNA.

One host cell useful in the present invention is a host cell stably transformed with the sequences encoding rep and cap, and which is transfected with the adenovirus E1, E2a, and E40RF6 DNA and a construct carrying the minigene as described above. Stable rep and/or cap expressing cell lines, such as B-50 (PCT/US98/19463), or those described in U.S. Pat. No. 5,658,785, may also be similarly employed. Another desirable host cell contains the minimum adenoviral DNA which is sufficient to express E4 ORF6.

The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods which provide the desired nucleotide sequence.

Introduction of the molecules (as plasmids or viruses) into the host cell may also be accomplished using techniques known to the skilled artisan and as discussed throughout the specification. In the preferred embodiment, standard transfection techniques are used, e.g., CaPO.sub.4 transfection or electroporation, and/or infection by hybrid adenovirus/AAV vectors into cell lines such as the human embryonic kidney cell line HEK 293 (a human kidney cell line containing functional adenovirus El genes which provides transacting E1 proteins). Thus produced, the rAAV may be used to prepare the compositions and kits described herein, and used in the method of the invention.

2.1.1 AAV Vector Constructs

AAV vector constructs are constructed using known techniques to at least provide, as operatively linked components in the direction of transcription, (a) control elements including a transcriptional initiation region, (b) the DNA of interest, and (c) a transcriptional termination region. The control elements are selected to be functional in the targeted cell. The resulting construct which contains the operatively linked components is bounded (5′ and 3′) with functional AAV ITR sequences. The nucleotide sequences of AAV ITR regions are known. See, e.g., [99]; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides.

Additionally, AAV ITRs may be derived from any of several AAV serotypes, including AAV-1, AAV-2, AAV-3, AAV-4, AAV-5, AAV-6, AAV-7, AAV-8, etc. The 5′ and 3′ ITRs which flank a selected transgene expression cassette in an AAV vector plasmid need not necessarily be identical or derived from the same AAV serotype , as long as they function as intended, i.e., to allow for excision and replication of the bounded nucleotide sequence of interest when AAV rep gene products are present in the cell. Thus, rAAV vector design and production allows for exchanging of the capsid proteins between different AAV serotypes: Homologous vectors comprising an expression cassette flanked by e.g., AAV2-ITRs and packaged in an AAV2 capsid, can be produced as well as heterologous, hybrid vectors where the transgene expression cassette is flanked by e.g., AAV2 ITRs, but the capsid originates from another AAV serotype: The following combinations are feasible: rAAV2/1-8, where the first number defines the genome and the second the capsid of the AAV of origin. In its preferred embodiment, the gene transfer vector is produced using a rAAV2/5 design.

Suitable minigenes for use in AAV vectors will generally be less than about 5 kilobases (kb) in size, which is the case for TIMP-3 inducing minigenes comprising the TIMP-3 cDNA as transgene. The TIMP-3 cDNA might also include minor variations from the native human sequence, particularly those leading to conservative amino acid replacements that do not adversely affect TIMP-3 biological function.

The AAV sequences used in generating the minigenes, vectors, and capsids, and other constructs used in the present invention may be obtained from a variety of sources. For example, the sequences may be provided by AAV type 5, AAV type 2, AAV type 1, AAV type 3, AAV type 4, AAV type 6, or other AAV serotypes or other densoviruses. A variety of these viral serotypes and strains are available from the American Type Culture Collection, Manassas, Va., or are available from a variety of academic or commercial sources. Alternatively, it may be desirable to synthesize sequences used in preparing the vectors and viruses of the invention using known techniques, which may utilize AAV sequences which are published and/or available from a variety of databases. The source of the sequences utilized in preparation of the constructs of the invention is not a limitation of the present invention.

2.1.2 rAAV Virion Production

In order to produce rAAV virions, an AAV vector construct that has been constructed as described above is introduced into a suitable host cell using known techniques, such as by transfection. A number of transfection techniques are generally known in the art. See, e.g., [77, 78], Sambrook et al. (1989) Molecular Cloning, a laboratory manual, Cold Spring Harbor Laboratories, New York, Davis et al. (1986) Basic Methods in Molecular Biology, Elsevier, and [79]. Particularly suitable transfection methods include calcium phosphate co-precipitation [77], direct micro-injection into cultured cells [100], electroporation [101], liposome mediated gene transfer [102], lipid-mediated transduction [103], and nucleic acid delivery using high-velocity microprojectiles.

The AAV vector construct harboring the AAV minigene is preferably carried on a plasmid which is delivered to a host cell by transfection. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ AAV ITR) may contain sequences permitting replication of the AAV minigene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the AAV minigene is transfected into the cell, where it may exist transiently or as an episome. Alternatively, the AAV minigene (carrying the 5′ AAV ITR-heterologous molecule-3′ AAV ITR) may be stably integrated into a chromosome of the host cell. Suitable transfection techniques are known and may readily be utilized to deliver the AAV minigene to the host cell.

Generally, when delivering the AAV vector construct comprising the AAV minigene by transfection, the vector is delivered in an amount from about 5 .mu.g to about 100 .mu.g DNA, and preferably about 10 to about 50 .mu.g DNA to about 1.times.10.sup.4 cells to about 1.times.10.sup.13 cells, and preferably about 10.sup.5 cells. However, the relative amounts of vector DNA to host cells may be adjusted, taking into consideration such factors as the selected vector, the delivery method and the host cells selected.

For the purposes of the invention, suitable host cells for producing rAAV virions include microorganisms, yeast cells, insect cells, and mammalian cells, that can be, or have been, used for transfection. The term includes the progeny of the original cell which has been transfected. Thus, a “host cell” as used herein generally refers to a cell which has been transfected with an exogenous DNA sequence. Cells from the stable human cell line, 293 (readily available through, e.g., the ATCC under Accession No. ATCC CRL1573) are preferred in the practice of the present invention. Particularly, the human cell line 293 is a human embryonic kidney cell line that has been transformed with adenovirus type-5 DNA fragments [104], and expresses the adenoviral E1a and E1b genes [105]. The 293 cell line is readily transfected, and provides a particularly convenient platform in which to produce rAAV virions.

The components required to be cultured in the host cell to package the AAV minigene in the AAV capsid may be provided to the host cell in trans. Alternatively, any one or more of the required components (e.g., minigene, rep sequences, cap sequences, and/or accessory functions) may be provided by a stable host cell which has been engineered to contain one or more of the required components using methods known to those of skill in the art.

The minigene, rep sequences, cap sequences, and accessory (helper) functions required for producing the rAAV of the invention may be delivered to the packaging host cell in the form of any genetic element, e.g., naked DNA, a plasmid, phage, transposon, cosmid, virus, etc. which transfer the sequences carried thereon. The selected genetic element may be delivered by any suitable method, including transfection, electroporation, liposome delivery, membrane fusion techniques, high velocity DNA-coated pellets, viral infection and protoplast fusion.

2.1.3 AAV Packaging Functions

Host cells containing the above described AAV vector constructs must be rendered capable of providing AAV packaging functions in order to replicate and encapsidate the nucleotide sequences flanked by the AAV ITRs to produce rAAV virions. AAV packaging functions are generally AAV-derived coding sequences which can be expressed to provide AAV gene products that, in turn, function in trans for productive AAV replication and genome encapsidation. AAV packaging functions are used herein to complement necessary AAV functions that are missing from the AAV vectors. Thus, AAV packaging functions include one, or both of the major AAV ORFs, namely the rep and cap coding regions, or functional homologues thereof.

By “AAV rep coding region” is meant the art-recognized region of the AAV genome which encodes the replication proteins Rep78, Rep68, Rep52 and Rep40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., [99, 106]. Suitable homologues of the AAV rep coding region include the human herpesvirus 6 (HHV-6) rep gene which is also known to mediate AAV-2 DNA replication [107].

By “AAV cap coding region” is meant the art-recognized region of the AAV genome which encodes the capsid proteins VP1, VP2, and VP3, or functional homologues thereof. These cap expression products supply the packaging functions which are collectively required for packaging the viral genome. For a description of the AAV cap coding region, see, e.g., [99, 106].

AAV packaging functions are introduced into the host cell by transfecting the host cell with an AAV packaging construct either prior to, or concurrently with, the transfection of the AAV vector construct. AAV packaging constructs are thus used to provide at least transient expression of AAV rep and/or cap genes to complement missing AAV functions that are necessary for productive AAV infection. AAV packaging constructs lack AAV ITRs and can neither replicate nor package themselves. These constructs can be in the form of a plasmid, phage, transposon, cosmid, virus, or virion. A number of AAV packaging constructs have been described, such as the commonly used plasmids pAAV/Ad and pIM29+45 which encode both Rep and Cap expression products. See, e.g., [108, 109]. A number of other vectors have been described which encode Rep and/or Cap expression products. See, e.g., U.S. Pat. No. 5,139,941.

Additionally, when pseudotyping an AAV vector in an AAV5 capsid, the sequences encoding each of the essential Rep proteins may be supplied by the same AAV serotype as the ITRs, or the sequences encoding the Rep proteins may be supplied by different, but cross-reactive, AAV serotypes (e.g., AAV1, AAV2, AAV3, AAV4 and AAV6). For example, the Rep78/68 sequences may be from AAV2, whereas the Rep52/40 sequences may from AAV1.

In one embodiment, the host cell stably contains the capsid ORF under the control of a suitable promoter, such as those described above. Most desirably, in this embodiment, the capsid ORF is expressed under the control of an inducible promoter. In another embodiment, the capsid ORF is supplied to the host cell in trans. When delivered to the host cell in trans, the capsid ORF may be delivered via a plasmid which contains the sequences necessary to direct expression of the selected capsid ORF in the host cell. Most desirably, when delivered to the host cell in trans, the plasmid carrying the capsid ORF also carries other sequences required for packaging the rAAV, e.g., the rep sequences.

In another embodiment, the host cell stably contains the rep sequences under the control of a suitable promoter, such as those described above. Most desirably, in this embodiment, the essential Rep proteins are expressed under the control of an inducible promoter. In another embodiment, the rep ORF is supplied to the host cell in trans. When delivered to the host cell in trans, the rep ORF may be delivered via a plasmid which contains the sequences necessary to direct expression of the selected rep ORF in the host cell. Most desirably, when delivered to the host cell in trans, the plasmid carrying the rep ORF also carries other sequences required for packaging the rAAV, e.g., the cap sequences.

Thus, in one embodiment, the rep and cap sequences may be transfected into the host cell on a single nucleic acid molecule and exist in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

Optionally, the rep and/or cap sequences may be supplied on a vector that contains other DNA sequences that are to be introduced into the host cells. For instance, the vector may contain the rAAV vector construct comprising the AAV minigene. The vector may comprise one or more of the genes encoding the helper functions, e.g., the adenoviral proteins E1, E2a, and E40RF6, and the gene for VA RNA.

In another embodiment, the promoter for rep is an inducible promoter, as discussed above in connection with regulatory sequences and promoters. One preferred promoter for rep expression is the T7 promoter. The vector comprising the rep gene regulated by the T7 promoter and the cap gene, is transfected or transduced into a cell which either constitutively or inducibly expresses the T7 polymerase. See WO 98/10088, published Mar. 12, 1998.

Preferably, the promoter used in the AAV packaging construct may be any of the constitutive, inducible or native promoters known to one of skill in the art or as discussed above. In one embodiment, an AAV p5 promoter sequence is employed. The selection of the AAV to provide any of these sequences does not limit the invention.

The spacer is an optional element in the design of the AAV packaging construct. The spacer is a DNA sequence interposed between the promoter and the rep gene ATG start site. The spacer may have any desired design; that is, it may be a random sequence of nucleotides, or alternatively, it may encode a gene product, such as a marker gene. The spacer may contain genes which typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls. Two exemplary sources of spacer sequences are the X phage ladder sequences or yeast ladder sequences, which are available commercially, e.g., from Gibco or Invitrogen, among others. The spacer may be of any size sufficient to reduce expression of the rep78 and rep68 gene products, leaving the rep52, rep40 and cap gene products expressed at normal levels. The length of the spacer may therefore range from about 10 bp to about 10.0 kbp, preferably in the range of about 100 bp to about 8.0 kbp. To reduce the possibility of recombination, the spacer is preferably less than 2 kbp in length; however, the invention is not so limited.

Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. The methods employed for constructing embodiments of this invention are conventional genetic engineering or recombinant engineering techniques such as those described in the references above. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

2.1.4 AAV Accessory Functions

The host cell (or packaging cell) must also be rendered capable of providing non AAV derived functions, or “accessory functions”, in order to produce rAAV virions. Accessory functions are non AAV derived viral and/or cellular functions upon which AAV is dependent for its replication. Thus, accessory functions include at least those non AAV proteins and RNAs that are required in AAV replication, including those involved in activation of AAV gene transcription, stage specific AAV mRNA splicing, AAV DNA replication, synthesis of rep and cap expression products and AAV capsid assembly. Viral-based accessory functions can be derived from any of the known helper viruses.

Particularly, accessory functions can be introduced into and then expressed in host cells using methods known to those of skill in the art. Commonly, accessory functions are provided by infection of the host cells with an unrelated helper virus. A number of suitable helper viruses are known, including adenoviruses, Herpes viruses such as Herpes Simplex Virus types 1 and 2, and vaccinia viruses. Non-viral accessory functions will also find use herein, such as those provided by cell synchronization using any of various known agents [110-112]. Alternatively and preferentially, accessory functions can be provided using an accessory function vector construct. Accessory function vector constructs include nucleotide sequences that provide one or more accessory functions. An accessory function vector is capable of being introduced into a suitable host cell in order to support efficient AAV virion production in the host cell. Accessory function vectors can be in the form of a plasmid, phage, virus, transposon or cosmid. Accessory vector constructs can also be in the form of one or more linearized DNA or RNA fragments which, when associated with the appropriate control elements and enzymes, can be transcribed or expressed in a host cell to provide accessory functions.

Nucleic acid sequences providing the accessory functions can be obtained from natural sources, such as from the genome of adenovirus (especially Adenovirus serotype 5), or constructed using recombinant or synthetic methods known in the art. In this regard, adenovirus-derived accessory functions have been widely studied, and a number of adenovirus genes involved in accessory functions have been identified and partially characterized. See, e.g., Carter, B. J. (1990) “Adeno-Associated Virus Helper Functions,” in CRC Handbook of Parvoviruses, vol. I (P. Tijssen, ed.), and [106]. Specifically, early adenoviral gene regions E1a, E2a, E4, VAI RNA and, possibly, E1b are thought to participate in the accessory process [113]. Herpes Virus-derived accessory functions have been described as well [114]. Vaccinia virus-derived accessory functions have also been described [110].

Most desirably, the necessary accessory functions are provided from an adenovirus source. In one embodiment, the host cell is provided with and/or contains an E1a gene product, an E1b gene product, an E2a gene product, and/or an E4 ORF6 gene product. The host cell may contain other adenoviral genes such as VAI RNA, but these genes are not required. In a preferred embodiment, no other adenovirus genes or gene functions are present in the host cell. The DNA sequences encoding the adenovirus E4 ORF6 genes and the E1 genes and/or E2a genes useful in this invention may be selected from among any known adenovirus type, including the presently identified 46 human types [see, e.g., American Type Culture Collection]. Similarly, adenoviruses known to infect other animals may supply the gene sequences. The selection of the adenovirus type for each E1, E2a, and E4 ORF6 gene sequence does not limit this invention. The sequences for a number of adenovirus serotypes, including that of serotype Ad5, are available from Genbank. A variety of adenovirus strains are available from the American Type Culture Collection (ATCC), Manassas, Va., or are available by request from a variety of commercial and institutional sources. Any one or more of human adenoviruses Types 1 to 46 may supply any of the adenoviral sequences, including E1, E2a, and/or E4 ORF6.

The adenovirus E1a, E1b, E2a, and/or E40RF6 gene products, as well as any other desired accessory functions, can be provided using any means that allows their expression in a cell. Each of the sequences encoding these products may be on a separate vector, or one or more genes may be on the same vector. The vector may be any vector known in the art or disclosed above, including plasmids, cosmids and viruses. Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously-added factors, for example.

As a consequence of the infection of the host cell with a helper virus, or transfection of the host cell with an accessory function vector construct, accessory functions are expressed which transactivate the AAV packaging construct to produce AAV Rep and/or Cap proteins. The Rep expression products direct excision of the recombinant DNA (including the DNA of interest) from the AAV vector construct. The Rep proteins also serve to replicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as CsCI gradients or column purification. Further, if helper virus infection is employed to express the accessory functions, residual helper virus can be inactivated, using known methods. For example, adenovirus can be inactivated by heating to temperatures of approximately 60.degree. C. for, e.g., 20 minutes or more. This treatment selectively inactivates the helper virus which is heat labile, while preserving the rAAV which is heat stable. The resulting rAAV virions are then ready for use for DNA delivery to a variety of target cells.

2.2 In vivo Delivery of rAAV Virions and Pharmaceutical Compositions

The present invention relates to a method for the transfer of nucleic acid compositions to the cells of an individual. The method comprises the step of contacting cells of said individual with nucleic acid transfer vectors which include said nucleic acid compositions, thereby delivering said nucleic acid compositions to the nucleus within said cells. The nucleic acid composition transfer vectors are administered to the cells of said individual on an in vivo basis, i.e., the contact with the cells of the individual takes place within the body of the individual in accordance with the procedures which are most typically employed. In its preferred embodiment, the nucleic acid transfer vector is a recombinant AAV virion.

The nucleic acid transfer vector is preferably suspended in a pharmaceutically acceptable delivery vehicle (i.e., physiologically compatible carrier), for administration to a human or non-human mammalian patient. Suitable carriers may be readily selected by one of skill in the art and may depend on the nature of the nucleic acid transfer vector chosen. Pharmaceutical compositions will comprise sufficient genetic material to produce a therapeutically effective amount of TIMP-3. The pharmaceutical compositions will also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. Other exemplary carriers include lactose, sucrose, calcium phosphate, gelatin, dextran, agar, pectin, peanut oil, sesame oil, and water. The selection of the carrier is not a limitation of the present invention. Optionally, the compositions of the invention may contain, in addition to the nucleic acid transfer vector (for example, rAAV) and carrier(s), other conventional pharmaceutical ingredients, such as preservatives, or chemical stabilizers. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin and albumin. A thorough discussion of pharmaceutically acceptable excipients is available in REMINGTON'S PHARMACEUTICAL SCIENCES (Mack Pub. Co., N.J. 1991).

Appropriate doses will depend, among other factors, on the specifics of the transfer vector chosen, on the route of administration, on the mammal being treated (e.g., human or non-human primate or other mammal), age, weight, and general condition of the subject to be treated, the severity of the cancer being treated, the location of the cancer being treated and the mode of administration. Thus, the appropriate dosage may vary from patient to patient. An appropriate effective amount can be readily determined by one of skill in the art. In one specific embodiment, the nucleic acid transfer vector is an AAV2/5 hybrid vector. A therapeutically effective human dosage for in vivo delivery of said vector according to the present invention is believed to be in the range of from about 20 to about 50 ml of saline solution containing concentrations of from about 10¹⁰ to 10¹⁴ functional vector/ml solution. The dosage will be adjusted to balance the therapeutic benefit against any side effects. In yet another embodiment, pharmaceutically effective dose of the rAAV is generally in the range of concentrations of from about 1.times.10.sup.5 to 1.times.10.sup.50 genomes rAAV, about 10.sup.8 to 10.sup.20 genomes rAAV, about 10.sup.10 to about 10.sup.16 genomes, or about 10.sup.11 to 10.sup.16 genomes rAAV. A preferred human dosage may be about 1.times.10.sup.13 AAV genomes rAAV. Such concentrations may be delivered in about 0.001 ml to 100 ml, 0.05 to 50 ml, or 10 to 25 ml of a carrier solution. Other effective dosages can be readily established by one of ordinary skill in the art through routine trials establishing dose response curves.

Dosage treatment may be a single dose schedule or a multiple dose schedule. Moreover, the subject may be administered as many doses as appropriate. One of skill in the art can readily determine an appropriate number of doses.

However, the dosage may need to be adjusted to take into consideration an alternative route of administration, or balance the therapeutic benefit against any side effects. Such dosages may vary depending upon the therapeutic application for which the recombinant vector is employed. The levels of expression of the transgene can be monitored to determine the frequency of dosage of viral vectors, preferably AAV vectors, containing the minigene.

The vector particles are administered in sufficient amounts to enter the desired cells and to guarantee sufficient levels of functionality of the transferred nucleic acid composition to provide a therapeutic benefit without undue adverse, or with medically acceptable, physiological effects which can be determined by those skilled in the medical arts. In its preferred embodiment, conventional and pharmaceutically acceptable routes of non-invasive in vivo administration to the lung include without limitation direct delivery to the target organ, tissue or site, by intranasal, or inhaling administration. Routes of administration may be combined, if desired.

In some embodiments, conventional pharmaceutically acceptable routes of administration of rAAV may be combined in a regimen which includes delivery by inhalation as described above. These routes include, but are not limited to, direct delivery to the liver, intravenous, intramuscular, subcutaneous, intradermal, oral and other parental routes of administration. Such regimens may involve delivery of the transgene product prior to, or subsequent to, delivery by inhalation according to the present invention.

Optionally, in specific embodiments, rAAV-mediated delivery according to the invention may be combined with delivery by other viral and non-viral vectors. Such other viral vectors including, without limitation, adenoviral vectors, retroviral vectors, lentiviral vectors herpes simplex virus (HSV) vectors, and baculovirus vectors may be readily selected and generated according to methods known in the art. Similarly, non-viral vectors, including, without limitation, liposomes, lipid-based vectors, polyplex vectors, molecular conjugates, polyamines and polycation vectors, may be readily selected and generated according to methods known in the art. When administered by these alternative routes, the dosage is desirable in the range described above.

In one embodiment, the gene transfer vector is a rAAV and the route of administration is inhalation with lung cells as target cells. In that instance, when prepared for use as an inhalant, the pharmaceutical compositions are prepared as fluid unit doses using the rAAV and a suitable pharmaceutical vehicle for delivery by an atomizing spray pump, or by dry powder for insufflation. For use as aerosols, the rAAV can be packaged in a pressurized aerosol container together with a gaseous or liquefied propellant, for example, dichlorodifluormethane, carbon dioxide, nitrogen, propane, and the like, with the usual components such as cosolvents and wetting agents, as may be necessary or desirable. A pharmaceutical kit of said embodiment, desirably contains a container for oral or intranasal inhalation, which delivers a metered dose in one, two, or more actuations. Suitably, the kit also contains instructions for use of the spray pump or other delivery device, instructions on dosing, and an insert regarding the active agent (i.e., the transgene and/or rAAV). A single actuation of a pump spray or inhaler generally delivers contains in the range of about 10.sup.5 to about 10.sup.15 genome copies (GC), about 10.sup.8 to about 10.sup.12, and/or about 10.sup.10 GC, in a liquid containing 10 .mu.g to 250 .mu.g carrier, 25 .mu.g to 100 .mu.g, or 40 .mu.g to 50 .mu.g, carrier. Suitably, a dose is delivered in one or two actuations. However, other suitable delivery methods may be readily determined. The doses may be repeated daily, weekly, or monthly, for a predetermined length of time or as prescribed.

2.3 Malignancies

Malignancies and related disorders that can be treated or prevented by administration of a Therapeutic of the invention include, but are not limited to, those disorders listed below (for a review of such disorders, see Fishman et al., 1985, Medicine, 2d Ed., J.B. Lippencott Co., Philadelphia):

Solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, Kaposi's sarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, virally induced cancers.

In specific embodiments, a Therapeutic of the invention is used to treat a neoplasm such as a gestational trophoblastic tumor, or testicular germ cell tumor, or cancer of the bladder, pancreas, cervix, lung, liver, ovary, colon or stomach, or adenocarcinoma or a virally induced cancer. In a preferred embodiment, a Therapeutic of the invention is used to treat and/or prevent lung carcinoma or small cell lung carcinoma. In another embodiment, a Therapeutic of the invention is used to treat carcinoma of the breast or prostate.

In one aspect of the invention, the Therapeutic is administered in conjunction with other cancer therapy, such as chemotherapy (e.g., treatment with adriamycin, bleomycin, vincristine, vinblastine, doxorubicin and/or Taxol). The efficacy of a Therapeutic against a particular cancer can be determined by any method known in the art.

A Therapeutic of the invention can also be administered to treat premalignant conditions and to prevent progression to a neoplastic or malignant state, including but not limited to those disorders listed above. Such prophylactic or therapeutic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions [1]. Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of a Therapeutic of the invention. Such characteristics of a transformed phenotype include morphology changes, looser substratum attachment, loss of contact inhibition, loss of anchorage dependence, protease release, increased sugar transport, decreased serum requirement, expression of fetal antigens, etc. In other embodiments, a patient which exhibits one or more of the following predisposing factors for malignancy is treated by administration of an effective amount of a Therapeutic: Familial polyposis or Gardner's syndrome (possible forerunners of colon cancer) or a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurotibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; [1]. In another specific embodiment, a Therapeutic of the invention is administered to a human patient to prevent progression to breast, colon, lung, pancreatic, or uterine cancer, or melanoma or sarcoma.

### 3. EMBODIMENTS

In its preferred embodiment, the present invention provides a rAAV virion comprising a nucleic acid composition which comprises a TIMP-3 inducing minigene and AAV2 ITRs. Said rAAV virion is pseudotyped in a capsid of AAV serotype 5 (AAV5)—resulting in an AAV2/5 virion.

In its preferred embodiment, the invention involves infecting the lung cells of a patient in vivo via inhalation of a composition composed of a rAAV comprising a TIMP-3 inducing expression cassette and AAV5 capsid proteins. The use of rAAV derived from AAV5 capsids is particularly desirable, as they allow for long-term gene expression as compared to other vectors which transduce lung cells efficiently (e.g., adenoviral vectors). Additionally, rAAV having capsids derived from AAV5 or a fragment thereof transduce lung cells in a manner which allows for secretion of proteins into the blood stream (in contrast to adenoviral vectors which secrete into the lumen of the lung rather than into the blood stream).

In its preferred embodiment, TIMP-3 expression within the transduced target cell and/or TIMP-3 secretion from the transduced target cell is achieved by transducing said target cell with a gene transfer vector comprising a nucleic acid composition which comprises the TIMP-3 coding sequence and all the regulatory sequences necessary for its transcription and translation in said target cell.

In another embodiment, TIMP-3 expression within the transduced target cell and/or TIMP-3 secretion from the transduced target cell is achieved by transducing said target cell with a gene transfer vector comprising a nucleic acid composition which comprises the coding sequence for a naturally occurring or artificial transcription factor and all the regulatory sequences necessary for its transcription and translation in said target cell. Upon expression of said transcription factor, said transcription factor will induce or upregulate by itself or in combination with other internal or external transcription factors, the expression of the endogenous TIMP-3 gene to therapeutically effective levels. Those of ordinary skill in the art can—without undue effort—create such artificial transcription factors [80-84] as to induce or upregulate transcription and thus expression of TIMP-3.

In yet another embodiment, TIMP-3 expression within the transduced target cell and/or TIMP-3 secretion from the transduced target cell is achieved by transducing said target cell with two or more gene transfer vectors—preferentially rAAV vectors—where each transfer vectors comprises a nucleic acid composition which comprises a partial TIMP-3 coding sequence. Transduction of the same target cell with the different gene transfer vectors—preferentially rAAV vectors—will lead to the restoration of a functional TIMP-3 transcription and/or translation unit by one of two mechanisms: (1) intermolecular recombination and/or (2) trans-splicing.

(1) Intermolecular Recombination

In one embodiment, the TIMP-3 coding sequence is split into two parts and incorporated into two independent gene transfer vectors with an overlapping region for homologous recombination on the DNA level. This homology region comprises 100 base pairs, more than 100 base pairs or less than 100 base pairs. For example, gene transfer vector A might contain (5′ to 3′): AAV-ITR—promoter—5′ TIMP-3 ORF (nucleotides 1-400)—AAV-ITR; gene transfer vector B might contain (5′ to 3′): AAV-ITR—3′ TIMP-3 ORF (nucleotides 250—TIMP-3 stop codon)—SV40 poly(A)—AAV-ITR. Upon successful transduction of the target cell with both gene transfer vectors, a complete TIMP-3 transcriptional unit can be generated through homologous recombination on the DNA level within the homologous TIMP-3 region, resulting in: AAV-ITR—promoter—TIMP-3 ORF—SV40 poly(A)—AAV-ITR. Thus, transcription of a functional RNA resulting in the translation into TIMP-3 protein can be achieved. How to generate gene transfer vectors for intermolecular recombination has been published in prior art [115].

(2) Trans-Splicing

In another embodiment, the TIMP-3 coding sequence is split into two parts and incorporated into two independent gene transfer vectors with appropriate splice signals incorporated into each gene transfer vector, preferentially rAAV-based vectors. For example, gene transfer vector A might contain (5′ to 3′): AAV-ITR—promoter—5′ TIMP-3 ORF (nucleotides 1-400)—Splice Donor—AAV-ITR; gene transfer vector B might contain (5′ to 3′): AAV-ITR—Splice Acceptor—3′ TIMP-3 ORF (nucleotides 250—TIMP-3 stop codon)—SV40 poly(A)—AAV-ITR. Upon successful transduction of the target cell with both gene transfer vectors, a complete TIMP-3 transcriptional unit can be generated through homologous recombination on the DNA level within the homologous ITR sequences, resulting in: AAV-ITR—promoter—5′ TIMP-3 ORF—Splice Donor—recombined AAV—ITR—Splice Acceptor—3′ TIMP3 ORF—SV40 poly(A)—AAV-ITR. Thus, transcription of a pre-mRNA and subsequent splicing will result in a functional TIMP-3 mRNA whose translation results in expression of a functional TIMP-3 protein. How to generate gene transfer vectors for trans-splicing has been published in prior art [72-74].

Similar to multi-vector based expression of TIMP-3, the expression of transcription factors leading to the expression of TIMP-3 can be achieved with a similar multi-vector approach. This would also be within the scope of this invention. Moreover, the expression of any protein—from a single- or multi-vector system—that either directly or indirectly leads to the expression and/or secretion of TIMP-3 and/or a substantially homologoues protein upon transduction of a target cell will be within the scope of this invention.

In one embodiment, the method of the invention involves transducing the lung cells of a patient in vivo via inhalation of a composition composed of a rAAV comprising the TIMP-3 coding sequence under the control of sequences which direct expression thereof and AAV5 capsid proteins. As defined herein, AAV5 capsid proteins include hybrid capsid proteins which contain a functional portion of the AAV5 capsid. This embodiment of the invention which uses rAAV with a serotype 5 capsid protein is particularly desirable, because AAV5 capsids are not recognized by neutralizing antibodies to other AAV serotypes. In addition, AAV5 capsids have been found to have tissue tropism for lung cells. However, the methods and compositions of the invention are not limited to rAAV derived from AAV5. One of skill in the art can readily select other rAAV vectors for use in the present invention.

In another embodiment, the invention provides a rAAV virion comprising a nucleic acid composition which comprises a TIMP-3 inducing minigene and AAV ITRs. In said embodiment, both the AAV ITRs and capsid proteins are of the same serotype. In one example, a rAAV containing AAV5 ITRs and a TIMP-3-inducing minigene and an AAV5 capsid, the rAAV contains modified 5′ and/or 3′ ITRs, as described below. However, the selection of the AAV serotypes for the TIMP-3 inducing minigene and/or AAV capsid are not a limitation of the present invention. In another embodiment, the invention provides a rAAV virus, in which both the AAV ITRs and capsid protein are of serotype 5. In this embodiment, the virus preferably contains modified 5′ and/or 3′ ITRs. More particularly, the virus desirably contains a 175-bp 5∝ITR and a 182-bp 3′ ITR. Desirably, in this embodiment, the rAAV5 virus further contains a promoter and an intron upstream of the transgene, and a woodchuck hepatitis virus post-transcriptional element and a bovine growth hormone polyA signal downstream of the transgene.

In yet another embodiment, the invention provides a rAAV virion, in which both the AAV ITRs and capsid protein are independently selected from among AAV serotypes, including, without limitation, AAV1, AAV2, AAV3, AAV4, AAV5, and AAV6. For example, the invention may utilize a rAAV1 vector, a rAAV2 vector, a rAAV2/1 vector, and rAAV1/2 vector and/or a rAAV2/5 vector, as desired.

In another embodiment, the invention provides a method of infecting a selected host cell in vivo with a rAAV containing an AAV5 transfer vector packaged in a capsid protein of another AAV serotype by inhalation. Optionally, a sample from the host may be first assayed for the presence of antibodies to a selected AAV serotype. A variety of assay formats for detecting neutralizing antibodies are well known to those of skill in the art. The selection of such an assay is not a limitation of the present invention [116, 117]. The results of this assay may be used to determine from which serotype the capsid protein will be preferred for delivery, e.g., by the absence of neutralizing antibodies specific for that capsid serotype.

In another embodiment of this method, the delivery of vector with an AAV5 capsid protein may precede or follow delivery of a heterologous molecule (e.g., gene) via a vector with a different serotype AAV capsid protein. Thus, delivery via multiple rAAV vectors may be used for repeat delivery of a desired molecule to a selected host cell. Desirably, subsequently administered rAAV carry the same minigene as the first rAAV vector, but the subsequently administered vectors contain capsid proteins of serotypes which differ from the first vector. For example, if a first rAAV has an AAV5 capsid protein, subsequently administered rAAV may have capsid proteins selected from among the other serotypes, including AAV2, AAV1, AAV3A, AAV3B, AAV4 and AAV6.Alternatively, if a first rAAV has an AAV2 capsid protein, subsequently administered rAAV may have an AAV5 capsid. Still other suitable combinations will be readily apparent to one of skill in the art.

### 4. DESCRIPTION OF THE PREFERRED EMBODIMENT

In its preferred embodiment, the present invention provides a rAAV virion comprising a nucleic acid composition which comprises a TIMP-3 inducing minigene and AAV2 ITRs. Said rAAV virion is pseudotyped in a capsid of AAV serotype 5 (AAV5)—resulting in an AAV2/5 virion. TIMP-3 expression within the transduced target cell and/or TIMP-3 secretion from the transduced target cell is achieved by transducing said target cell with said AAV2/5 virion comprising a nucleic acid composition which comprises the TIMP-3 coding sequence and all the regulatory sequences necessary for its transcription and translation in said target cell with said target cell being a lung cell. Lung cells will be infected in vivo via inhalation or nasal instillation.

The use of rAAV derived from AAV5 capsids is particularly desirable, as they allow for long-term gene expression as compared to other vectors which transduce lung cells efficiently (e.g., adenoviral vectors). Additionally, rAAV having capsids derived from AAV5 or a fragment thereof transduce lung cells in a manner which allows for secretion of proteins into the blood stream (in contrast to adenoviral vectors which secrete into the lumen of the lung rather than into the blood stream).

In its preferred embodiment, the expression of TIMP-3 is driven by the CMV promoter. The complete sequence of the preferred Therapeutic (AAV2/5—CMV—TIMP-3) is as follows (5′ to 3′):

5′ agcgcccaatacgcaaaccgcctctccccgcgcgttggccgattcattaatgcagctggcacgacaggtttcccg
actggaaagcgggcagtgagcgcaacgcaattaatgtgagttagctcactcattaggcaccccaggctttacacttt
atgcttccggctcgtatgttgtgtggaattgtgagcggataacaatttcacacaggaaacagctatgaccatgatta
cgccagatttaattaaggctgcgcgctcgctcgctcactgaggccgcccgggcaaagcccgggcgtcgggcgacctt
tggtcgcccggcctcagtgagcgagcgagcgcgcagagagggagtggccaactccatcactaggggttccttgtagt
taatgattaacccgccatgctacttatctacgtagccatgctctaggaagatcggaattcgcccttaagctagctag
ttattaatagtaatcaattacggggtcattagttcatagcccatatatggagttccgcgttacataacttacggtaa
atggcccgcctggctgaccgcccaacgacccccgcccattgacgtcaataatgacgtatgttcccatagtaacgcca
atagggactttccattgacgtcaatgggtggagtatttacggtaaactgcccacttggcagtacatcaagtgtatca
tatgccaagtacgccccctattgacgtcaatgacggtaaatggcccgcctggcattatgcccagtacatgaccttat
gggactttcctacttggcagtacatctacgtattagtcatcgctattaccatggtgatgcggttttggcagtacatc
aatgggcgtggatagcggtttgactcacggggatttccaagtctccaccccattgacgtcaatgggagtttgttttg
gcaccaaaatcaacgggactttccaaaatgtcgtaacaactccgccccattgacgcaaatgggcggtaggcgtgtac
ggtgggaggtctatataagcagagctggtttagtgaaccgtcagatcctgcagaagttggtcgtgaggcactgggca
ggtaagtatcaaggttacaagacaggtttaaggagaccaatagaaactgggcttgtcgagacagagaagactcttgc
gtttctgataggcacctattggtcttactgacatccactttgcctttctctccacaggtgtccaggcggccgccatg
accccttggctcgggctcatcgtgctcctgggcagctggagcctgggggactggggcgccgaggcgtgcacatgctc
gcccagccacccccaggacgccttctgcaactccgacatcgtgatccgggccaaggtggtggggaagaagctggtaa
aggaggggcccttcggcacgctggtctacaccatcaagcagatgaagatgtaccgaggcttcaccaagatgccccat
gtgcagtacatccacacggaagcttccgagagtctctgtggccttaagctggaggtcaacaagtaccagtacctgct
gacaggtcgcgtctatgatggcaagatgtacacggggctgtgcaacttcgtggagaggtgggaccagctcaccctct
cccagcgcaaggggctgaactatcggtatcacctgggttgtaactgcaagatcaagtcctgctactacctgccttgc
tttgtgacttccaagaacgagtgtctctggaccgacatgctctccaatttcggttaccctggctaccagtccaaaca
ctacgcctgcatccggcagaagggcggctactgcagctggtaccgaggatgggcccccccggataaaagcatcatca
atgccacagacccctgaagcttggatccaatcaacctctggattacaaaatttgtgaaagattgactggtattctta
actatgttgctccttttacgctatgtggatacgctgctttaatgcctttgtatcatgctattgcttcccgtatggct
ttcattttctcctccttgtataaatcctggttgctgtctctttatgaggagttgtggcccgttgtcaggcaacgtgg
cgtggtgtgcactgtgtttgctgacgcaacccccactggttggggcattgccaccacctgtcagctcctttccggga
ctttcgctttccccctccctattgccacggcggaactcatcgccgcctgccttgcccgctgctggacaggggctcgg
ctgttgggcactgacaattccgtggtgttgtcggggaagctgacgtcctttccatggctgctcgcctgtgttgccac
ctggattctgcgcgggacgtccttctgctacgtcccttcggccctcaatccagcggaccttccttcccgcggcctgc
tgccggctctgcggcctcttccgcgtcttcgagatctgcctcgactgtgccttctagttgccagccatctgttgttt
gcccctcccccgtgccttccttgaccctggaaggtgccactcccactgtcctttcctaataaaatgaggaaattgca
tcgcattgtctgagtaggtgtcattctattctggggggtggggtggggcaggacagcaagggggaggattgggaaga
caatagcaggcatgctggggactcgagttaagggcgaattcccgattaggatcttcctagagcatggctacgtagat
aagtagcatggcgggttaatcattaactacaaggaacccctagtgatggagttggccactccctctctgcgcgctcg
ctcgctcactgaggccgggcgaccaaaggtcgcccgacgcccgggctttgcccgggcggcctcagtgagcgagcgag
cgcgcagccttaattaacctaattcactggccgtcgttttacaacgtcgtgactgggaaaaccctggcgttacccaa
cttaatcgccttgcagcacatccccctttcgccagctggcgtaatagcgaagaggcccgcaccgatcgcccttccca
acagttgcgcagcctgaatggcgaatgggacgcgccctgtagcggcgcattaagcgcggcgggtgtggtggttacgc
gcagcgtgaccgctacacttgccagcgccctagcgcccgctcctttcgctttcttcccttcctttctcgccacgttc
gccggctttccccgtcaagctctaaatcgggggctccctttagggttccgatttagtgctttacggcacctcgaccc
caaaaaacttgattagggtgatggttcacgtagtgggccatcgccccgatagacggtttttcgccctttgacgctgg
agttcacgttcctcaatagtggactcttgttccaaactggaacaacactcaaccctatctcggtctattcttttgat
ttataagggatttttccgatttcggcctattggttaaaaaatgagctgatttaacaaaaatttaacgcgaattttaa
caaaatattaacgtttataatttcaggtggcatctttcggggaaatgtgcgcggaacccctatttgtttatttttct
aaatacattcaaatatgtatccgctcatgagacaataaccctgataaatgcttcaataatattgaaaaaggaagagt
atgagtattcaacatttccgtgtcgcccttattcccttttttgcggcattttgccttcctgtttttgctcacccaga
aacgctggtgaaagtaaaagatgctgaagatcagttgggtgcacgagtgggttacatcgaactggatctcaatagtg
gtaagatccttgagagttttcgccccgaagaacgttttccaatgatgagcacttttaaagttctgctatgtggcgcg
gtattatcccgtattgacgccgggcaagagcaactcggtcgccgcatacactattctcagaatgacttggttgagta
ctcaccagtcacagaaaagcatcttacggatggcatgacagtaagagaattatgcagtgctgccataaccatgagtg
ataacactgcggccaacttacttctgacaacgatcggaggaccgaaggagctaaccgcttttttgcacaacatgggg
gatcatgtaactcgccttgatcgttgggaaccggagctgaatgaagccataccaaacgacgagcgtgacaccacgat
gcctgtagtaatggtaacaacgttgcgcaaactattaactggcgaactacttactctagcttcccggcaacaattaa
tagactggatggaggcggataaagttgcaggaccacttctgcgctcggcccttccggctggctggtttattgctgat
aaatctggagccggtgagcgtgggtctcgcggtatcattgcagcactggggccagatggtaagccctcccgtatcgt
agttatctacacgacggggagtcaggcaactatggatgaacgaaatagacagatcgctgagataggtgcctcactga
ttaagcattggtaactgtcagaccaagtttactcatatatactttagattgatttaaaacttcatttttaatttaaa
aggatctaggtgaagatcctttttgataatctcatgaccaaaatcccttaacgtgagttttcgttccactgagcgtc
agaccccgtagaaaagatcaaaggatcttcttgagatcctttttttctgcgcgtaatctgctgcttgcaaacaaaaa
aaccaccgctaccagcggtggtttgtttgccggatcaagagctaccaactctttttccgaaggtaactggcttcagc
agagcgcagataccaaatactgtccttctagtgtagccgtagttaggccaccacttcaagaactctgtagcaccgcc
tacatacctcgctctgctaatcctgttaccagtggctgctgccagtggcgataagtcgtgtcttaccgggttggact
caagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcgga
caggtatccggtaagcggcagggtcggaacaggagagcgcacgagggagcttccagggggaaacgcctggtatcttt
atagtcctgtcgggtttcgccacctctgacttgagcgtcgatttttgtgatgctcgtcaggggggcggagcctatgg
aaaaacgccagcaacgcggcctttttacggttcctggccttttgctgcggttttgctcacatgttctttcctgcgtt
atcccctgattctgtggataaccgtattaccgcctttgagtgagctgataccgctcgccgcagccgaacgaccgagc
gcagcgagtcagtgagcgaggaagcggaag-3′

The artisan will be able to reconstruct AAV2/5—CMV—TIMP-3 from the sequence information provided. As a control, an rAAV virion expressing lacZ as transgene (AAV 2/5—CMV—lacZ), described was used as lacZ does not possess anti-cancer activity. The sequence and cloning of AAV 2/5—CMV—lacZ has been described in prior art [71].

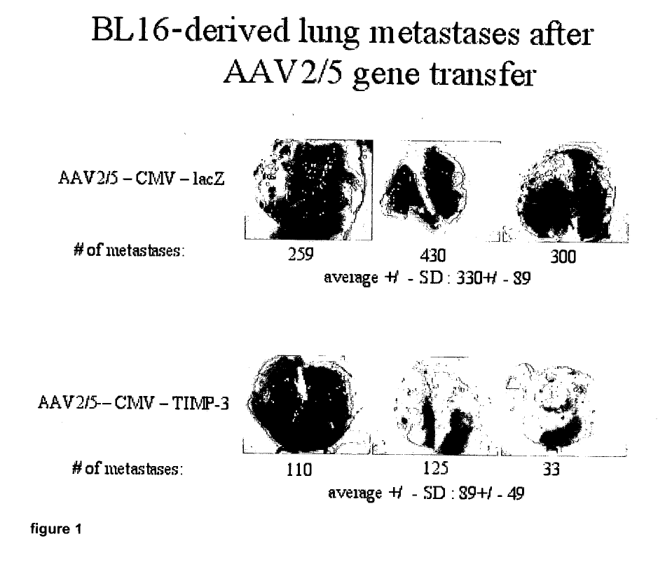

Both AAV2/5—CMV—TIMP-3 and AAV2/5—CMV—lacZ were prepared by triple transfection and purified by CsCI gradients as described herein and in prior art [71]. Physical titers were assessed by Real Time PCR. Six week old C57/BL6 mice (Charles River Italia, Calco, LC, Italy) were intranasally administered with 2.5×10e11 particles of AAV2/5—CMV—TIMP-3 and AAV2/5—CMV—lacZ, respectively: Five animals were used in the therapeutic (TIMP-3) and control (lacZ) groups. Four weeks after initial vector administration, each animal received a tail vein injection of 7.5×10e4 BL16 murine melanoma cells. 2 weeks later animals were anesthetized, perfused with 4% paraformaldehyde, their lungs harvested and the number of melanoma-derived lung metastases counted.

Whereas mice in the control (lacZ) group had on average 330±89 lung metastases, the TIMP-3 treated mice had on average only 89±49 lung metastases, a result that is statistically significant (p<0.05; see FIG. 1). Thus, gene transfer induced expression of TIMP-3 (here: by means of AAV2/5—CMV—TIMP-3) inhibited the growth of cancer (here: lung metastases).

Although the present invention has been described with reference to specific embodiments, numerous modifications and variations can be made and still the result will come within the scope of the invention. No limitation with respect to the specific embodiments disclosed herein is intended or should be inferred.

### PRIOR ART

### U.S. Patent Documents

| | | |
|---|---|---|
| 6,531,456 | Kurtzman et al. | March 2003 |
| 5,854,019 | Sedlacek et al. | December 1998 |
| 5,219,740 | | |
| 5,658,785 | | |
| 5,139,941 | | |

References

1. Angell, R., Basic Pathology. 2nd edition ed. 1976: W. B. Saunders Co. 68-122.

2. Burrows, F. J. and P. E. Thorpe, Vascular targeting—a new approach to the therapy of solid tumors. Pharmacol Ther, 1994. 64(1): p. 155-74.

3. Baxter, L. T. and R. K. Jain, Transport of fluid and macromolecules in tumors. III. Role of binding and metabolism. Microvasc Res, 1991. 41(1): p. 5-23.

4. Baxter, L. T. and R. K. Jain, Transport of fluid and macromolecules in tumors. IV. A microscopic model of the perivascular distribution. Microvasc Res, 1991. 41(2): p. 252-72.

5. Cristofanilli, M., C. Charnsangavej, and G. N. Hortobagyi, Angiogenesis modulation in cancer research: novel clinical approaches. Nat Rev Drug Discov, 2002. 1(6): p. 415-26.

6. Ferrara, N. and K. Alitalo, Clinical applications of angiogenic growth factors and their inhibitors. Nat Med, 1999. 5(12): p. 1359-64.

7. Tosetti, F., et al., ‘Angioprevention’: angiogenesis is a common and key target for cancer chemopreventive agents. Faseb J, 2002. 16(1): p. 2-14.

8. Eriksson, U. and K. Alitalo, VEGF receptor 1 stimulates stem-cell recruitment and new hope for angiogenesis therapies. Nat Med, 2002. 8(8): p. 775-7.

9. Cawston, T., et al., Role of TIMP and MMP inhibition in preventing connective tissue breakdown. Ann N Y Acad Sci, 1994. 732: p. 75-83.

10. Brew, K., D. Dinakarpandian, and H. Nagase, Tissue inhibitors of metalloproteinases: evolution, structure and function. Biochim Biophys Acta, 2000. 1477(1-2): p. 267-83.

11. Gomez, D. E., et al., Tissue inhibitors of metalloproteinases: structure, regulation and biological functions. Eur J Cell Biol, 1997. 74(2): p. 111-22.

12. Hashimoto, T., et al., Abnormal Expression of Matrix Metalloproteinases and Tissue Inhibitors of Metalloproteinases in Brain Arteriovenous Malformations * Growth and Bleeding in BAVM: Another Role for MMPs. Stroke, 2003. 34(4): p. 925-31.

13. Nagase, H. and K. Brew, Engineering of tissue inhibitor of metalloproteinases mutants as potential therapeutics. Arthritis Res, 2002. 4 Suppl 3: p. S51-61.

14. Woessner, J. F., Jr., That impish TIMP: the tissue inhibitor of metalloproteinases-3. J Clin Invest, 2001. 108(6): p. 799-800.

15. Woessner, J. F., Jr., MMPs and TIMPs. An historical perspective. Methods Mol Biol, 2001. 151: p. 1-23.

16. Anand-Apte, B., et al., A review of tissue inhibitor of metalloproteinases-3 (TIMP-3) and experimental analysis of its effect on primary tumor growth. Biochem Cell Biol, 1996. 74(6): p. 853-62.

17. Tan, H. K., et al., Tissue inhibitor of metalloproteinase 1 inhibits excitotoxic cell death in neurons. Mol Cell Neurosci, 2003. 22(1): p. 98-106.

18. Ahonen, M., et al., Tissue inhibitor of metalloproteinases-3 induces apoptosis in melanoma cells by stabilization of death receptors. Oncogene, 2003. 22(14): p. 2121-34.

19. Mannello, F. and G. Gazzanelli, Tissue inhibitors of metalloproteinases and programmed cell death: conundrums, controversies and potential implications. Apoptosis, 2001.6(6): p.479-82.

20. Isashiki, Y. and N. Ohba, [Sorsby's fundus dystrophy: a literature review]. Nippon Ganka Gakkai Zasshi, 1999. 103(1): p. 3-11.

21. Baker, A. H., et al., Inhibition of invasion and induction of apoptotic cell death of cancer cell lines by overexpression of TIMP-3. Br J Cancer, 1999. 79(9-10): p.1347-55.

22. Ahonen, M., A. H. Baker, and V. M. Kahari, Adenovirus-mediated gene delivery of tissue inhibitor of metalloproteinases-3 inhibits invasion and induces apoptosis in melanoma cells. Cancer Res, 1998. 58(11): p. 2310-5.

23. Ahonen, M., et al., Antitumor activity and bystander effect of adenovirally delivered tissue inhibitor of metalloproteinases-3. Mol Ther, 2002. 5(6): p. 705-15.

24. Spurbeck, W. W., et al., Enforced expression of tissue inhibitor of matrix metalloproteinase-3 affects functional capillary morphogenesis and inhibits tumor growth in a murine tumor model. Blood, 2002.100(9): p. 3361-8.

25. Spurbeck, W. W., et al., Retroviral vector-producer cell-mediated in vivo gene transfer of TIMP-3 restricts angiogenesis and neuroblastoma growth in mice. Cancer Gene Ther, 2003. 10(3): p. 161-7.

26. Ennis, B. W. and L. M. Matrisian, Matrix degrading metalloproteinases. J Neurooncol, 1994. 18(2): p. 105-9.

27. Sternlicht, M. D. and Z. Werb, How matrix metalloproteinases regulate cell behavior. Annu Rev Cell Dev Biol, 2001.17: p. 463-516.

28. Sang, Q. X., Complex role of matrix metalloproteinases in angiogenesis. Cell Res, 1998. 8(3): p. 171-7.

29. Hofmann, U. B., et al., Matrix metalloproteinases in human melanoma. J Invest Dermatol, 2000. 115(3): p. 337-44.

30. Matrisian, L. M., The matrix-degrading metalloproteinases. Bioessays, 1992. 14(7): p. 455-63.

31. Matrisian, L. M., Matrix metalloproteinase gene expression. Ann N Y Acad Sci, 1994. 732: p. 42-50.

32. Stetler-Stevenson, W. G., S. Aznavoorian, and L. A. Liotta, Tumor cell interactions with the extracellular matrix during invasion and metastasis. Annu Rev Cell Biol, 1993. 9: p. 541-73.

33. Stetler-Stevenson, W. G., L. A. Liotta, and D. E. Kleiner, Jr., Extracellular matrix 6: role of matrix metalloproteinases in tumor invasion and metastasis. Faseb J, 1993. 7(15): p. 1434-41.

34. Margulies, I.M ., et al., Urinary type IV collagenase: elevated levels are associated with bladder transitional cell carcinoma. Cancer Epidemiol Biomarkers Prev, 1992. 1(6): p. 467-74.

35. Stearns, M. E. and M. Wang, Immunoassays of the metalloproteinase (MMP-2) and tissue inhibitor of metalloproteinase (TIMP 1 and 2) levels in noninvasive and metastatic PC-3 clones: effects of taxol. Oncol Res, 1994. 6(4-5): p. 195-201.

36. Bode, W., et al., Insights into MMP-TIMP interactions. Ann N Y Acad Sci, 1999. 878: p. 73-91.

37. McCachren, S. S., Expression of metalloproteinases and metalloproteinase inhibitor in human arthritic synovium. Arthritis Rheum, 1991. 34(9): p. 1085-93.

38. Saarialho-Kere, U. K., Patterns of matrix metalloproteinase and TIMP expression in chronic ulcers. Arch Dermatol Res, 1998. 290 Suppl: p. S47-54.

39. Qi, J. H., et al., A novel function for tissue inhibitor of metalloproteinases-3 (TIMP3): inhibition of angiogenesis by blockage of VEGF binding to VEGF receptor-2. Nat Med, 2003. 9(4): p. 407-15.

40. Denekamp, J. and B. Littbrand, [Angiogenesis—a new field for cancer therapy]. Lakartidningen, 1991. 88(45): p. 3788-90.

41. Denekamp, J., Inadequate vasculature in solid tumours: consequences for cancer research strategies. BJR Suppl, 1992. 24: p. 111-7.

42. Denekamp, J., Review article: angiogenesis, neovascular proliferation and vascular pathophysiology as targets for cancer therapy. Br J Radiol, 1993. 66(783): p. 181-96.

43. Denekamp, J., A. Dasu, and A. Waites, Vasculature and microenvironmental gradients: the missing links in novel approaches to cancer therapy? Adv Enzyme Regul, 1998. 38: p. 281-99.

44. Bicknell, R. and A. L. Harris, Anticancer strategies involving the vasculature: vascular targeting and the inhibition of angiogenesis. Semin Cancer Biol, 1992. 3(6): p. 399-407.

45. Gagliardi, A., H. Hadd, and D. C. Collins, Inhibition of angiogenesis by suramin. Cancer Res, 1992. 52(18): p. 5073-5.

46. Thorpe, P. E., et al., Heparin-steroid conjugates: new angiogenesis inhibitors with antitumor activity in mice. Cancer Res, 1993. 53(13): p. 3000-7.

47. Yamaoka, M., et al., Angiogenesis inhibitor TNP-470 (AGM-1470) potently inhibits the tumor growth of hormone-independent human breast and prostate carcinoma cell lines. Cancer Res, 1993. 53(21): p. 5233-6.

48. Yamaoka, M., et al., Inhibition of tumor growth and metastasis of rodent tumors by the angiogenesis inhibitor O-(chloroacetyl-carbamoyl)fumagillol (TNP-470; AGM-1470). Cancer Res, 1993. 53(18): p. 4262-7.

49. Olson, K. A., et al., A monoclonal antibody to human angiogenin suppresses tumor growth in athymic mice. Cancer Res, 1994. 54(17): p. 4576-9.

50. O'Reilly, M. S., et al., Angiostatin: a novel angiogenesis inhibitor that mediates the suppression of metastases by a Lewis lung carcinoma. Cell, 1994. 79(2): p. 315-28.

51. O'Reilly, M. S., et al., Angiostatin: a circulating endothelial cell inhibitor that suppresses angiogenesis and tumor growth. Cold Spring Harb Symp Quant Biol, 1994. 59: p. 471-82.

52. Scarpa, M., et al., Characterization of recombinant helper retroviruses from Moloney-based vectors in ecotropic and amphotropic packaging cell lines. Virology, 1991. 180(2): p. 849-52.

53. Miller, A. D. and G. J. Rosman, Improved retroviral vectors for gene transfer and expression. Biotechniques, 1989. 7(9): p. 980-2, 984-6, 989-90.

54. Miller, A. D., Retrovirus packaging cells. Hum Gene Ther, 1990. 1(1): p. 5-14.

55. Burns, J. C., et al., Vesicular stomatitis virus G glycoprotein pseudotyped retroviral vectors: concentration to very high titer and efficient gene transfer into mammalian and nonmammalian cells. Proc Natl Acad Sci U S A, 1993. 90(17): p. 8033-7.

56. Boris-Lawrie, K. A. and H. M. Temin, Recent advances in retrovirus vector technology. Curr Opin Genet Dev, 1993. 3(1): p. 102-9.

57. Li, Z., et al., Murine leukemia induced by retroviral gene marking. Science, 2002. 296(5567): p. 497.

58. Hacein-Bey-Abina, S., et al., A serious adverse event after successful gene therapy for X-linked severe combined immunodeficiency. N Engl J Med, 2003. 348(3): p. 255-6.

59. Haj-Ahmad, Y. and F. L. Graham, Development of a helper-independent human adenovirus vector and its use in the transfer of the herpes simplex virus thymidine kinase gene. J Virol, 1986. 57(1): p. 267-74.

60. Mittereder, N., et al., Evaluation of the efficacy and safety of in vitro, adenovirus-mediated transfer of the human cystic fibrosis transmembrane conductance regulator cDNA. Hum Gene Ther, 1994. 5(6): p. 717-29.

61. Barr, E., et al., Efficient catheter-mediated gene transfer into the heart using replication-defective adenovirus. Gene Ther, 1994. 1 (1): p. 51-8.

62. Bett, A. J., L. Prevec, and F. L. Graham, Packaging capacity and stability of human adenovirus type 5 vectors. J Virol, 1993. 67(10): p. 5911-21.

63. Berkner, K. L., Development of adenovirus vectors for the expression of heterologous genes. Biotechniques, 1988. 6(7): p. 616-29.

64. Seth, P., Adenovirus-dependent release of choline from plasma membrane vesicles at an acidic pH is mediated by the penton base protein. J Virol, 1994. 68(2): p. 1204-6.

65. Rich, D. P., et al., Development and analysis of recombinant adenoviruses for gene therapy of cystic fibrosis. Hum Gene Ther, 1993. 4(4): p. 461-76.

66. Lai, C. M., Y. K. Lai, and P. E. Rakoczy, Adenovirus and adeno-associated virus vectors. DNA Cell Biol, 2002. 21(12): p. 895-913.

67. Owens, R. A., Second generation adeno-associated virus type 2-based gene therapy systems with the potential for preferential integration into AAVS1. Curr Gene Ther, 2002. 2(2): p. 145-59.

68. Paterna, J. C. and H. Bueler, Recombinant adeno-associated virus vector design and gene expression in the mammalian brain. Methods, 2002. 28(2): p. 208-1 8.

69. Nicklin, S. A. and A. H. Baker, Tropism-modified adenoviral and adeno-associated viral vectors for gene therapy. Curr Gene Ther, 2002. 2(3): p. 273-93.

70. Chiorini, J. A., S. Afione, and R. M. Kotin, Adeno-associated virus (AAV) type 5 Rep protein cleaves a unique terminal resolution site compared with other AAV serotypes. J Virol, 1999. 73(5): p. 4293-8.

71. Hildinger, M., et al., Hybrid vectors based on adeno-associated virus serotypes 2 and 5 for muscle-directed gene transfer. J Virol, 2001. 75(13): p. 6199-203.

72. Reich, S. J., et al., Efficient trans-splicing in the retina expands the utility of adeno-associated virus as a vector for gene therapy. Hum Gene Ther, 2003. 14(1): p. 37-44.

73. Duan, D., Y. Yue, and J. F. Engelhardt, Expanding AAV packaging capacity with trans-splicing or overlapping vectors: a quantitative comparison. Mol Ther, 2001. 4(4): p. 383-91.

74. Yan, Z., et al., Trans-splicing vectors expand the utility of adeno-associated virus for gene therapy. Proc Natl Acad Sci U S A, 2000. 97(12): p. 6716-21.

75. lzraeli, S. and G. Rechavi, Molecular medicine—an overview. Isr Med Assoc J, 2002. 4(8): p. 638-40.

76. Mohr, L. and M. Geissler, [Gene therapy: new developments]. Schweiz Rundsch Med Prax, 2002. 91(51-52): p. 2227-35.

77. Graham, F. L. and A. J. van der Eb, A new technique for the assay of infectivity of human adenovirus 5 DNA. Virology, 1973. 52(2): p. 456-67.

78. Graham, F. L. and A. J. van der Eb, Transformation of rat cells by DNA of human adenovirus 5. Virology, 1973. 54(2): p. 536-9.

79. Chu, G. and P. A. Sharp, SV40 DNA transfection of cells in suspension: analysis of efficiency of transcription and translation of T-antigen. Gene, 1981. 13(2): p. 197-202.

80. Lee, D. K., W. Seol, and J. S. Kim, Custom DNA-Binding Proteins and Artificial Transcription Factors. Curr Top Med Chem, 2003. 3(6): p. 645-57.

81. Segal, D. J. and C. F. Barbas, 3rd, Custom DNA-binding proteins come of age: polydactyl zinc-finger proteins. Curr Opin Biotechnol, 2001. 12(6): p. 632-7.

82. Beerli, R. R. and C. F. Barbas, 3rd, Engineering polydactyl zinc-finger transcription factors. Nat Biotechnol, 2002. 20(2): p. 135-41.

83. Imanishi, M., et al., Design of novel zinc finger proteins: towards artificial control of specific gene expression. Eur J Pharm Sci, 2001. 13(1): p. 91-7.

84. Nagaoka, M. and Y. Sugiura, Artificial zinc finger peptides: creation, DNA recognition, and gene regulation. J lnorg Biochem, 2000. 82(1-4): p. 57-63.

85. Wang, L., et al., Sustained correction of bleeding disorder in hemophilia B mice by gene therapy. Proc Natl Acad Sci U S A, 1999. 96(7): p. 3906-10.

86. Graham, R. M., M. Friedman, and G. W. Hoyle, Sensory nerves promote ozone-induced lung inflammation in mice. Am J Respir Crit Care Med, 2001. 164(2): p. 307-13.

87. Ehrhardt, A., et al., Development of pulmonary bronchiolo-alveolar adenocarcinomas in transgenic mice overexpressing murine c-myc and epidermal growth factor in alveolar type II pneumocytes. Br J Cancer, 2001. 84(6): p. 813-8.

88. Palmarini, M., et al., The long terminal repeat of Jaagsiekte sheep retrovirus is preferentially active in differentiated epithelial cells of the lungs. J Virol, 2000. 74(13): p. 5776-87.

89. Borok, Z., et al., Differential regulation of rat aquaporin-5 promoter/enhancer activities in lung and salivary epithelial cells. J Biol Chem, 2000. 275(34): p. 26507-14.

90. Boshart, M., et al., A very strong enhancer is located upstream of an immediate early gene of human cytomegalovirus. Cell, 1985. 41(2): p. 521-30.

91. No, D., T. P. Yao, and R. M. Evans, Ecdysone-inducible gene expression in mammalian cells and transgenic mice. Proc Natl Acad Sci U S A, 1996. 93(8): p. 3346-51.

92. Gossen, M. and H. Bujard, Tight control of gene expression in mammalian cells by tetracycline-responsive promoters. Proc Natl Acad Sci U S A, 1992. 89(12): p. 5547-51.

93. Gossen, M., et al., Transcriptional activation by tetracyclines in mammalian cells. Science, 1995. 268(5218): p. 1766-9.

94. Harvey, D. M. and C. T. Caskey, Inducible control of gene expression: prospects for gene therapy. Curr Opin Chem Biol, 1998. 2(4): p. 512-8.

95. Wang, Y., et al., Ligand-inducible and liver-specific target gene expression in transgenic mice. Nat Biotechnol, 1997.15(3): p. 239-43.

96. Wang, Y., et al., Positive and negative regulation of gene expression in eukaryotic cells with an inducible transcriptional regulator. Gene Ther, 1997. 4(5): p. 432-41.

97. Magari, S. R., et al., Pharmacologic control of a humanized gene therapy system implanted into nude mice. J Clin Invest, 1997. 100(11): p. 2865-72.

98. Roux, K. H., Using mismatched primer-template pairs in touchdown PCR. Biotechniques, 1994. 16(5): p. 812-4.

99. Kotin, R. M., Prospects for the use of adeno-associated virus as a vector for human gene therapy. Hum Gene Ther, 1994. 5(7): p. 793-801.

100. Capecchi, M. R., High efficiency transformation by direct microinjection of DNA into cultured mammalian cells. Cell, 1980. 22(2 Pt 2): p. 479-88.

101. Shigekawa, K. and W. J. Dower, Electroporation of eukaryotes and prokaryotes: a general approach to the introduction of macromolecules into cells. Biotechniques, 1988. 6(8): p. 742-51.

102. Mannino, R. J. and S. Gould-Fogerite, Liposome mediated gene transfer. Biotechniques, 1988. 6(7): p. 682-90.

103. Felgner, P. L., et al., Lipofection: a highly efficient, lipid-mediated DNA-transfection procedure. Proc Natl Acad Sci U S A, 1987. 84(21): p. 7413-7.

104. Graham, F. L., et al., Characteristics of a human cell line transformed by DNA from human adenovirus type 5. J Gen Virol, 1977. 36(1): p. 59-74.

105. Aiello, L., et al., Adenovirus 5 DNA sequences present and RNA sequences transcribed in transformed human embryo kidney cells (HEK-Ad-5 or 293). Virology, 1979. 94(2): p. 460-9.

106. Muzyczka, N., Use of adeno-associated virus as a general transduction vector for mammalian cells. Curr Top Microbiol Immunol, 1992. 158: p. 97-129.

107. Thomson, B. J., et al., Human herpesvirus 6 (HHV-6) is a helper virus for adeno-associated virus type 2 (AAV-2) and the AAV-2 rep gene homologue in HHV-6 can mediate AAV-2 DNA replication and regulate gene expression. Virology, 1994. 204(1): p. 304-11.

108. Samulski, R. J., L. S. Chang, and T. Shenk, Helper-free stocks of recombinant adeno-associated viruses: normal integration does not require viral gene expression. J Virol, 1989. 63(9): p. 3822-8.

109. McCarty, D. M., M. Christensen, and N. Muzyczka, Sequences required for coordinate induction of adeno-associated virus p19 and p40 promoters by Rep protein. J Virol, 1991. 65(6): p. 2936-45.

110. Schlehofer, J. R., M. Ehrbar, and H. zur Hausen, Vaccinia virus, herpes simplex virus, and carcinogens induce DNA amplification in a human cell line and support replication of a helpervirus dependent parvovirus. Virology, 1986. 152(1): p. 110-7.

111. McPherson, R. A., L. J. Rosenthal, and J. A. Rose, Human cytomegalovirus completely helps adeno-associated virus replication. Virology, 1985. 147(1): p. 217-22.

112. Buller, R. M., et al., Herpes simplex virus types 1 and 2 completely help adenovirus-associated virus replication. J Virol, 1981. 40(1): p. 241-7.

113. Janik, J. E., M. M. Huston, and J. A. Rose, Locations of adenovirus genes required for the replication of adenovirus-associated virus. Proc Natl Acad Sci U S A, 1981. 78(3): p. 1925-9.

114. Young, J. F. and H. D. Mayor, Adeno-associated virus—an extreme state of viral defectiveness. Prog Med Virol, 1979. 25: p. 113-32.

115. Halbert, C. L., J. M. Allen, and A. D. Miller, Efficient mouse airway transduction following recombination between AAV vectors carrying parts of a larger gene. Nat Biotechnol, 2002. 20(7): p. 697-701.

116. Manning, W. C., et al., Transient immunosuppression allows transgene expression following readministration of adeno-associated viral vectors. Hum Gene Ther, 1998. 9(4): p. 477-85.

117. Fisher, K. J., et al., Recombinant adeno-associated virus for muscle directed gene therapy. Nat Med, 1997. 3(3): p. 306-12.

We claim:

1. A method of treating cancer, preventing cancer and/or inhibiting growth of cancer (cells) in a mammalian subject comprising administering to the subject a therapeutically effective amount of a nucleic acid composition in vivo, comprising:
(a) providing a gene transfer vector, wherein said gene transfer vector comprises at least one nucleic acid composition whose expression directly or indirectly leads to the expression and/or secretion of Tissue inhibitor of metalloproteinase-3 (TIMP-3, referenced by SEQ ID NO: 2), wherein said nucleic acid composition comprises expression control elements that comprise a heterologous promoter, wherein said promoter does not equal the TIMP-3 promoter nor is a cell cycle-regulated promoter module; and
(b) delivering said gene transfer vector to and/or within said mammalian subject wherein transduction of suitable target cells results in local (in the vicinity of the tumor or tumor bed) and/or systemic expression of said nucleic acid composition.

2. The method of claim 1, wherein the gene transfer vector is a viral vector.

3. The method of claim 1, wherein the gene transfer vector is a non-viral vector.

4. The method of claim 2, wherein the gene transfer vector is a recombinant adeno-associated viral (rAAV) vector.

5. The method of claim 4, wherein the rAAV vector is of serotype 5.

6. The method of claim 4, wherein the rAAV vector is of serotype 2/5.

7. The method of claim 2, wherein the gene transfer vector is a lentiviral vector.

8. The method of claim 2, wherein the gene transfer vector is an adenoviral vector.

9. The method of claim 2, wherein the gene transfer vector is a Herpes Simplex Virus-based vector.

10. The method of claim 1, wherein said nucleic acid composition encodes TIMP-3 (referenced by SEQ ID NO: 2) or a substantially homologous protein.

11. The method of claim 1, wherein said nucleic acid composition encodes a transcription factor or protein whose expression leads to the expression and/or secretion of TIMP-3 (referenced by SEQ ID NO: 2).

12. The method of claim 1, wherein several gene transfer vectors are used each comprising its own nucleic acid composition with the diverse nucleic acid compositions encoding in their entirety TIMP-3 (referenced by SEQ ID NO: 2), a protein substantially homologous to TIMP-3 (referenced by SEQ ID NO: 2), and/or a transcription factor or protein whose expression leads to the expression and/or secretion of TIMP-3 (referenced by SEQ ID NO: 2).

13. The method of claim 1, wherein TIMP-3 (referenced by SEQ ID NO: 2) or a substantially homologous protein is expressed and/or secreted locally.

14. The method of claim 1, wherein TIMP-3 (referenced by SEQ ID NO: 2) or a substantially homologous protein is expressed and/or secreted systemically.

15. The method of claim 1, wherein cancer is treated and/or growth of cancer cells is inhibited by antiangiogenic effects.

16. The method of claim 1, wherein the cancer is a solid tumor.

17. The method of claim 1, wherein the cancer is selected from the group consisting of lung cancer, breast cancer, colon cancer, gastric cancer, brain cancer, pancreatic cancer, liver cancer, prostate cancer and lymphoma.

18. The method of claim 1, wherein said suitable target cells to be transduced are cancer cells.

19. The method of claim 1, wherein said suitable target cells to be transduced are not cancer cells.

20. The method of claim 1, wherein the transduced cells are lung cells.

21. The method of claim 1, wherein the transduced cells are gut cells, muscle cells, intestinal cells, liver cells, pancreatic cells, hematopoietic cells, stem cells and/or brain cells.

22. A pharmaceutical preparation comprising a gene transfer vector comprising a nucleic acid composition as claimed in claim 1.

23. A pharmaceutical preparation as claimed in claim 22, wherein said preparation is suitable for and/or administered by intravenous administration.

24. A pharmaceutical preparation as claimed in claim 22, wherein said preparation is suitable for and/or administered by intraarterial administration.

25. A pharmaceutical preparation as claimed in claim 22, wherein said preparation is suitable for and/or administered by intracavity injection.

26. A pharmaceutical preparation as claimed in claim 22, wherein said preparation is suitable for and/or administered by injection into tissue.

27. A pharmaceutical preparation as claimed in claim 22, wherein said preparation is suitable for and/or administered by injection into gaps in tissue.

28. A pharmaceutical preparation as claimed in claim 22, wherein said preparation is suitable for and/or administered by local administration.

29. A pharmaceutical preparation as claimed in claim 22, wherein said preparation is suitable for and/or administered by inhalation and/or nasal instillation.
